(19) United States
(12) Patent Application Publication
(10) Pub. No.: US 2026/0331274 A1
(43) Pub. Date: Oct. 22, 2026

(54) SYSTEM

(71) Applicant: SoftBank Group Corp., Tokyo (JP)

(72) Inventor: Kaori ISHII, Tokyo (JP)

(73) Assignee: SoftBank Group Corp., Tokyo (JP)

(21) Appl. No.: 19/650,414

(22) Filed: Apr. 17, 2026

(30) Foreign Application Priority Data
Apr. 22, 2025 (JP) 2025-070241

(51) Int. Cl. G16H 40/63; G06F 40/295; G06F 40/35

(52) U.S. Cl. CPC G16H 40/63; G06F 40/295; G06F 40/35

(57) ABSTRACT

The present invention relates to a life support system for supporting an independent life of an elderly person and realizing peace of mind for a family member in a remote location, wherein a communication unit analyzes a dialogue with the elderly person using generative AI, and automatically executes life support in cooperation with various external services and IoT devices, such as shopping support, meal arrangement, cleaning, medical support, and entertainment, a health condition detection unit detects a health abnormality or an emergency from an utterance or sensor data and automatically notifies a family member or an administrative agency, a remote monitoring unit provides an activity history and a health condition to a family member in real time, and a settlement management unit collectively settles usage fees of all services by cashless payment.

### CROSS-REFERENCE TO RELATED APPLICATION

The present application claims priority to and incorporates by reference the entire contents of Japanese Patent Application No. 2025-070241 filed in Japan on Apr. 22, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

The problem to be solved by the present invention is to provide an improvement in the QOL of an elderly person and a safe and secure living environment while reducing the burden on a family member and society as a whole, by comprehensively and efficiently realizing various life support services, medical support, entertainment provision, monitoring of a health condition, automatic response in an emergency, and the like utilizing generative AI and robot technology, while enabling the elderly person to maintain an independent life and enabling a family member in a remote location to watch over the elderly person with peace of mind, in order to solve these problems in view of the current situation where, in a rapidly progressing aging society, while the number of elderly people requiring support in their daily lives due to aging, diseases, and the like is increasing, it becomes difficult for the children's generation to devote sufficient time and effort to nursing care and life support for their parents due to dual income, child-rearing, work, and the like, and as a result, there is a social problem that elderly people live alone in rural or urban areas, making it difficult not only to maintain daily life such as shopping, hospital visits, meals, cleaning, and health management, but also increasing risks such as sudden poor physical condition, accidents, and solitary death, and also, the children's generation cannot sufficiently grasp the living conditions and health conditions of their parents, bearing regret and mental burden due to delayed response in emergencies, and furthermore, the elderly themselves feel a psychological burden of not wanting to cause trouble to their children, and in addition, social problems such as a shortage of nursing care human resources, delays in emergency transportation, and a strain on medical resources are becoming more serious.

### SUMMARY OF THE INVENTION

Means for solving the problem according to the present invention constitutes an elderly life support system comprising a communication unit, a service linkage unit, a remote monitoring unit, a settlement management unit, and a health condition detection unit, wherein the communication unit has a function of analyzing a natural language dialogue with an elderly person in real time using generative AI to recognize a request and a physical condition change of the elderly person, the service linkage unit has a function of automatically selecting and executing various life support services in cooperation with a plurality of external services and IoT devices, such as shopping support, meal arrangement, cleaning service, medical support, and entertainment provision, based on the request acquired from the communication unit, the remote monitoring unit has a function of recording an activity history, an order history, a health condition, and a service usage status of the elderly person, and enabling a family member living apart to view these pieces of information online, the settlement management unit has a function of managing usage fees of all services provided through the service linkage unit so that the usage fees can be settled collectively by a cashless payment service, and the health condition detection unit has a function of analyzing utterance content, an activity log, and IoT sensor information of the elderly person using generative AI, automatically notifying a family member or an administrative agency when there is no movement or utterance for a certain period of time or when an abnormal health condition is detected, and arranging emergency transportation as necessary, thereby comprehensively and efficiently realizing independent life support for the elderly person, peace of mind for the family member, and reduction of nursing care burden on society as a whole.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor indicated by reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), among others.

In the following embodiments, a RAM (Random Access Memory) indicated by reference numeral is a memory where information is temporarily stored and used as a work memory by the processor.

In the following embodiments, a storage indicated by reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) indicated by reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

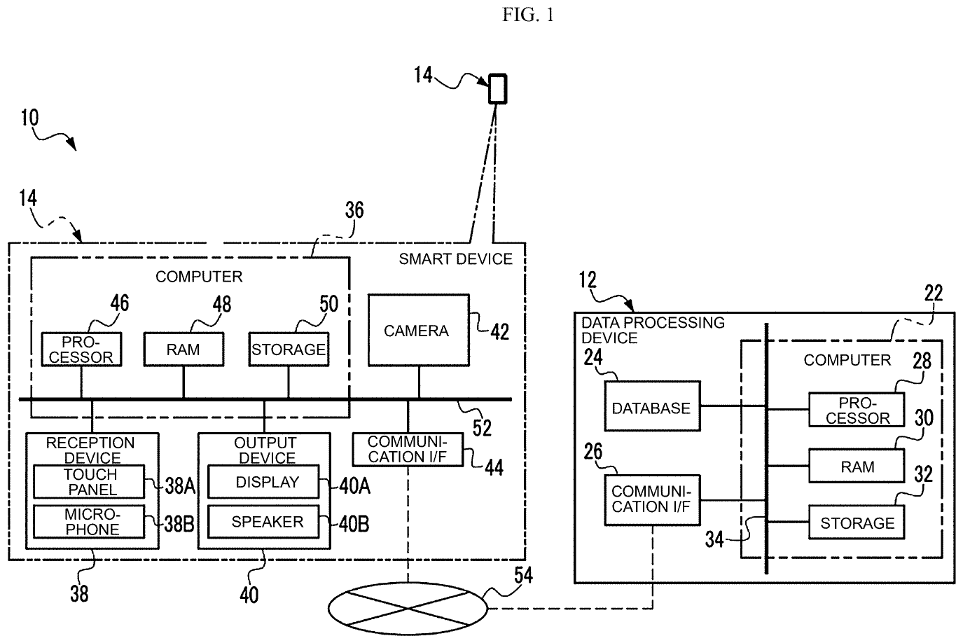

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, camera 42, and communication I/F 44 are also connected to the bus 52.

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A in the processor 46 sends data indicating user input accepted by the touch panel 38A and microphone 38B to the data processing device 12. In the data processing device 12, the specific processing unit 290 acquires data indicating user input.

The output device 40 comprises a display 40A and a speaker 40B, among others, and presents data to the user 20 by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

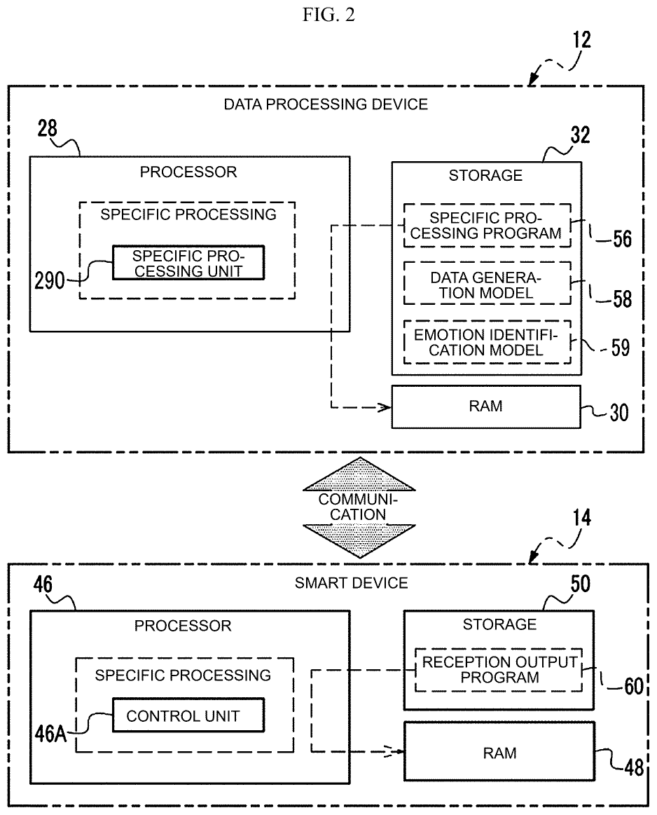

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart device 14, reception output processing is performed by the processor 46. The storage 50 stores a reception output program 60. The reception output program 60 is used in conjunction with the specific processing program 56 by the data processing system 10. The processor 46 reads the reception output program 60 from the storage 50 and executes it on the RAM 48. The reception output processing is realized by the processor 46 operating as a control unit 46A according to the reception output program 60 executed on the RAM 48. The smart device 14 may also have similar data generation models and emotion identification models as the data generation model 58 and emotion identification model 59, and perform the same processing as the specific processing unit 290 using these models. The reception output processing is realized by the processor 46 operating as the control unit 46A according to the reception output program 60 executed on the RAM 48.

Other devices besides the data processing device 12 may have the data generation model 58.

For example, a server device (e.g., a generation server) may have the data generation model 58.

In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1

The flow of the specific processing in Example 1 will be described. Each part of the system described below is realized by the data processing device 12 and the smart device 14. Additionally, the data processing device 12 is referred to as a “server,” and the smart device 14 is referred to as a “terminal.”

### Embodiment for Carrying Out the Present Invention

An embodiment of the present invention is a comprehensive life support system for supporting an independent life of an elderly person in a multifaceted manner and realizing peace of mind for family members living apart and a reduction in a nursing care burden on society as a whole, and adopts a distributed architecture using a server and a terminal. The terminal can take various forms such as a communication robot, a smart speaker, a tablet, a smartphone, a wearable device, and an IoT home appliance installed in a living space of the elderly person, and the server is installed on a cloud or in a data center of an operator. Specifically, the present system constructs a hierarchical distributed processing network that integrates edge computing and cloud computing. The present terminal executes primary data preprocessing using a built-in microprocessor and a local memory. The present terminal converts an acquired analog signal into digital data via an analog-to-digital conversion circuit. The present terminal applies a noise removal filtering process to improve data quality. The present terminal transmits the preprocessed data to the present server using a secure communication protocol. The present server includes a parallel computing cluster using a GPU. The present server applies an advanced machine learning model to a received large-scale data stream. The present distributed architecture suppresses consumption of a network bandwidth. The present distributed architecture reduces a calculation load on the server side. The present system enables a highly real-time response. The present system exhibits a technical effect of minimizing a communication delay. The present system is extremely useful in specific application fields such as monitoring of an elderly person and remote medical support in a smart home environment. The present system realizes an improvement in computer technology itself, which is optimization of traffic management and resource allocation of a computer network, rather than mere business efficiency improvement. The present system includes a failover function that dynamically switches a plurality of communication interfaces. The present system maintains availability of the system even in the event of a communication failure. The present system adopts a microservice architecture. The present system deploys each function as an independent container. The present system executes an autoscaling function that dynamically increases or decreases the number of containers in accordance with a load status. The present system maximizes a throughput of the entire system. The present system improves utilization efficiency of hardware resources. The present system replicates data to a plurality of nodes using a distributed database. The present system eliminates a single point of failure. The present system guarantees availability and durability of data. The present system implements end-to-end encrypted communication. The present system prevents eavesdropping and falsification of data. The present system executes strict access control based on a zero-trust security model. The present system permits access to the system only to a device and a user having a legitimate authority. The present system realizes advanced information processing by a cooperative operation of pure hardware and software different from human mental activity.

First, the communication unit acquires voice, video, an action, and a touch input of an elderly person through an interface such as a voice recognition microphone, a camera, a speaker, a touch panel, and a gesture sensor implemented on the terminal side, and transmits these pieces of data to the server in real time. On the server side, a natural language processing engine using generative AI operates, converts voice data into text, and performs multi-layered analysis such as semantic analysis of utterance content, emotion analysis, urgency determination, health condition estimation, and life pattern extraction. For example, it recognizes various utterances such as “I have a headache today” and “My legs hurt and I cannot move.” with high accuracy, such as “I want to go shopping”, “Please clean the room”, “I want to talk to my grandchild on a videophone”, “I want to listen to music”, “Tell me the news”, and “Call an ambulance”, and generates an optimal response and a service proposal by collating them with a context, a past utterance history, a life rhythm, and preference data. The generative AI dynamically performs individually optimized dialogue and proposal, for example, “Propose a fish dish because orders for fish dishes have decreased recently.” “Automatically propose cleaning because there is a tendency to desire cleaning every Monday”, “Prioritize proposing medical support because utterances of poor physical condition are increasing”, and “Propose a video call with a family member because utterances indicating a feeling of loneliness are increasing”. Specifically, the present communication unit digitizes an audio signal at a sampling frequency of 16 kHz and a quantization bit rate of 16 bits. The present communication unit converts an audio waveform into frequency domain data using a fast Fourier transform. The present communication unit applies a mel filter bank to extract mel-frequency cepstral coefficients as a feature vector. The present server inputs the extracted feature vector into a transformer-based large language model equipped with a self-attention mechanism. Input data to the present large language model is, for example, a time-series acoustic feature array converted from a voice saying “I have a headache today”. Input data to the present large language model is, for example, a text token sequence saying “I want to go shopping”. Input data to the present large language model is, for example, an acoustic tensor of an emergency utterance saying “Call an ambulance”. The present large language model converts the input feature vector into a 512-dimensional embedding tensor. The present large language model calculates a context-dependent hidden state vector through a multi-layer encoder and decoder network. The present large language model generates an appearance probability distribution of each word as an output. The present large language model outputs a classification label indicating an intention of an utterance as an output. The present large language model outputs a continuous value score from minus 1 to plus 1 indicating a polarity of an emotion as an output. A specific example of output data from the present large language model is, for example, an intention classification label of “medical support request”. A specific example of output data from the present large language model is, for example, a numerical score of “urgency 0.95”. A specific example of output data from the present large language model is, for example, a probability distribution vector of an emotion category of “sadness”. The present server inputs the output classification label and score to a subsequent rule-based inference engine. The present inference engine activates an emergency response module when the score exceeds a preset threshold. The present inference engine generates an appropriate response text based on the classification label. The present system converts the generated response text into audio waveform data by a speech synthesis engine. The present system transmits the audio waveform data to the present terminal to output it from the speaker. The present process dramatically improves accuracy of natural language processing. The present process exhibits a technical effect of providing high robustness even for an ambiguous utterance of an elderly person. The present system is used in specific application fields such as an interactive nursing care robot and an automatic response call center. The present large language model is pre-trained using a cross-entropy loss function. The present large language model optimizes a weight parameter using a gradient descent method. The present system executes a vector operation in a high-dimensional space in parallel on a GPU cluster. The present system realizes semantic analysis by a pure mathematical operation different from human mental activity. The present system stores a past dialogue history in a vector database. The present system searches for a past context most relevant to a current utterance using cosine similarity. The present system combines the searched context information with a prompt and inputs it to the large language model. The present system maintains consistency of a dialogue.

The service linkage unit is realized on the server side, and performs API linkage with various external services and IoT devices based on a request and a situation of an elderly person analyzed by generative AI. For example, in shopping support, it calls an API of an EC site to acquire a product list, proposes it to the elderly person by a display or voice on the terminal side, and automatically executes an ordering process after an order is confirmed. For example, when an utterance “I want to order milk and bread” is made, the generative AI also refers to a past purchasing history and an inventory status, lists necessary products, confirms an order, and automatically adjusts a delivery date and time. In meal support, it links with an API of a nearby restaurant or a meal delivery service, and collectively arranges from a meal menu proposal to ordering and delivery. For example, when an utterance “I want to eat Japanese food today” is made, the generative AI also considers a health condition, a past preference, and a nutritional balance, proposes an optimal menu, and executes an order. In a cleaning service, it calls an IoT control API of a robot vacuum cleaner to automatically operate it, or makes a reservation for a cleaning service by a person. For example, when an utterance “Please clean the living room” is made, it operates the robot vacuum cleaner and automatically reserves cleaning by a person as necessary. In medical support, it sets up a video call with a doctor using an API of a remote medical examination system, and arranges a pick-up and drop-off service of an autonomous vehicle as necessary. For example, when an utterance “I want to go to a hospital” or “I am running out of medicine” is made, the generative AI collectively executes a reservation of a medical institution, an arrangement of a pick-up and drop-off vehicle, and automatic delivery of medicine. In entertainment provision, it uses an API of a news distribution service or a music distribution service to realize functions such as “reading out the latest sports news”, “playing requested music”, and “acting as an agent for an event application”. For example, when an utterance “Tell me the baseball results” or “Play a song by my favorite singer” is made, the generative AI immediately acquires information and provides it as voice or video on the terminal. Specifically, the present service linkage unit communicates with an external system via a web API based on a RESTful architecture. The present service linkage unit generates a payload in a JSON format and transmits an HTTP request. The present service linkage unit acquires and manages a secure authentication token using an OAuth protocol. The present server executes a hybrid recommendation neural network combining collaborative filtering and content-based filtering. Input data to the present recommendation neural network is, for example, a multidimensional binary vector indicating a past purchasing history of a user. Input data to the present recommendation neural network is, for example, a feature matrix including attribute information of a product. Input data to the present recommendation neural network is, for example, a context tensor indicating a current time and weather. The present recommendation neural network inputs the input data to a multilayer perceptron to perform non-linear feature transformation. The present recommendation neural network generates a list of recommendation scores for each candidate item as an output. The present recommendation neural network provides an output value of a sigmoid function indicating a probability of matching a user's preference as an output. The present recommendation neural network outputs a probability distribution vector indicating a degree of interest by category as an output. A specific example of output data from the present recommendation neural network is, for example, a recommendation score of 0.92 for “milk”. A specific example of output data from the present recommendation neural network is, for example, a recommendation score of 0.88 for “Japanese bento”. A specific example of output data from the present recommendation neural network is, for example, a probability of 0.75 indicating a necessity of a “cleaning service”. The present server sorts the output recommendation scores in descending order. The present server extracts an item with a higher score and sets it as a request parameter to an external API. The present server analyzes a response from the external API to confirm an order completion status. The present process brings about an improvement in processing speed by automating service arrangement. The present process exhibits a technical effect of realizing a highly accurate proposal matching a user's preference. The present system is used in specific application fields such as a life infrastructure integration platform in a smart city. The present system guarantees ACID properties in transaction processing of a database. The present system maintains data consistency among a plurality of services using a distributed transaction coordinator. The present system executes a retry process using an exponential backoff algorithm when a network timeout or an error occurs. The present system improves fault tolerance of the system. The present system completely eliminates service arrangement by human manual work. The present system realizes autonomous negotiation between machines via a computer network.

The remote monitoring unit is mainly realized on the server side, but a dedicated application and a web interface are also provided to a terminal (such as a smartphone and a personal computer) used by a family member. On the server side, data such as an activity history, an order history, a health condition, and a service usage status of an elderly person are recorded and managed in chronological order, and generative AI analyzes these pieces of data to detect an abnormal tendency and a change in a life rhythm. For example, it automatically extracts signs such as “there has been no meal order for the past few days”, “an activity amount is significantly lower than usual”, and “words suggesting a poor physical condition are increasing in utterance content”, and transmits a push notification and an alert to a terminal of a family member. A family member can confirm “when and what a parent ordered”, “which service was used”, “a transition of a health condition”, “an emergency response history”, and the like in real time through an application or a web screen. Furthermore, it is also possible for a family member to remotely request an addition of a service, confirm a health condition, and give an emergency response instruction. For example, an instruction and confirmation such as “I want to request more cleaning this week”, “I want to reserve an accompaniment to a hospital visit next week”, and “I want to confirm a recent health condition in detail” are possible. Specifically, the present remote monitoring unit stores collected various data in a time-series database together with a time stamp. The present server executes an anomaly detection model using a long short-term memory network or a one-dimensional convolutional neural network. Input data to the present anomaly detection model is, for example, a time-series array having daily step counts for the past 30 days as elements. Input data to the present anomaly detection model is, for example, a numerical vector indicating a fluctuation in a heart rate every hour. Input data to the present anomaly detection model is, for example, a binary time-series tensor indicating presence or absence of a meal order. The present anomaly detection model encodes the input time-series data into a representation vector in a latent space. The present anomaly detection model predicts a future data value from a past data pattern. The present anomaly detection model calculates a mean square error between a predicted value and an actual observed value. The present anomaly detection model generates a calculated reconstruction error vector as an output. The present anomaly detection model outputs a continuous value score from 0 to 1 indicating a probability that an abnormality has occurred as an output. The present anomaly detection model outputs an attention weight vector indicating a contribution degree of a feature amount that caused an abnormality as an output. A specific example of output data from the present anomaly detection model is, for example, an abnormality probability score of 0.85 indicating a decrease in an activity amount. A specific example of output data from the present anomaly detection model is, for example, a reconstruction error value of 3.4 indicating a heart rate abnormality. A specific example of output data from the present anomaly detection model is, for example, a flag value of 1 indicating continuation of no meal order. The present server determines whether or not the output abnormality probability score exceeds a dynamically set threshold. The present server generates a push notification payload when the threshold is exceeded. The present server asynchronously transmits the generated payload to a terminal of a family member via a cloud messaging service. The present process discovers a minute change in a life rhythm early, which is impossible by human visual monitoring. The present process exhibits a technical effect of enabling data-driven preventive intervention. The present system is used in specific application fields such as a remote nursing care support system and a monitoring service for an elderly person living alone. The present anomaly detection model is trained by unsupervised learning using only data of a normal life pattern. The present anomaly detection model updates a weight of a network using a backpropagation algorithm. The present system executes a distance calculation in a high-dimensional time-series data space at high speed. The present system provides a technical improvement of autonomous pattern recognition and determination by a computer, rather than mere display of information. The present system establishes WebSocket communication in response to a request from a terminal of a family member. The present system realizes real-time bidirectional data streaming.

The settlement management unit is realized on the server side. It centrally aggregates usage fees of all services provided through the service linkage unit, and performs a collective settlement process in cooperation with an API of a cashless payment service. For example, even when a plurality of services such as shopping, a meal, cleaning, medical support, and entertainment are used, it automatically summarizes usage details and performs automatic withdrawal from a settlement account of an elderly person or a family member at the end of a month or every time of use. A settlement status and usage details can be confirmed at any time by a family member or an elderly person from a terminal. For example, it can immediately respond to a request such as “how much did food expenses cost this month”, “I want to confirm usage details of a medical service”, and “I want to confirm a payment status of a cleaning service”. Specifically, the present settlement management unit asynchronously receives billing event data transmitted from each service module via a message queuing system. The present settlement management unit temporarily caches the received event data in an in-memory database. The present settlement management unit periodically activates a batch processing program to aggregate the cached data. The present server converts the aggregated usage detail data into a structured JSON object. The present server generates a digital signature of the JSON object using a secure hash algorithm. The present server encrypts a communication path using a public key cryptosystem. The present server transmits a settlement request to an external settlement gateway API. Input data to a processing model in the present settlement management unit is, for example, an array of transaction records including a service ID and a usage amount. Input data to the processing model in the present settlement management unit is, for example, an encrypted character string including settlement account information of a user. Input data to the processing model in the present settlement management unit is, for example, a flag vector indicating presence or absence of use of a discount coupon or points. The present settlement management unit receives an HTTP status code indicating success or failure of a settlement process as an output. The present settlement management unit generates a response object including a unique transaction ID as an output. The present settlement management unit outputs a time stamp indicating a next scheduled billing date as an output. A specific example of output data from the present settlement management unit is, for example, a status code 200 indicating completion of a settlement. A specific example of output data from the present settlement management unit is, for example, an alphanumeric character string indicating a transaction ID. A specific example of output data from the present settlement management unit is, for example, a URL pointing to PDF data of an electronic receipt. The present server writes the output transaction ID into a settlement history table of a relational database. The present server transmits a notification of completion of a settlement to a user interface unit to update a screen display. The present process automates a complex billing calculation across a plurality of services. The present process exhibits a technical effect of eliminating a delay in a settlement process and ensuring certainty of a transaction. The present system is used in specific application fields such as an integrated subscription management platform and a common settlement infrastructure of a smart city. The present system prevents falsification of a transaction using a distributed ledger based on blockchain technology. The present system executes a smart contract to automatically transfer funds only when a condition is satisfied. The present system is an improvement in computer technology that not only substitutes for human accounting work but also provides advanced security based on a cryptographic proof. The present system performs concurrency control using a lock mechanism of a database. The present system completely prevents double billing and data inconsistency.

The health condition detection unit is realized on both the terminal side and the server side. On the terminal side, data acquired from a microphone, a camera, and various sensors (for example, an acceleration sensor, a heart rate sensor, a temperature sensor, a blood pressure sensor, a sleep sensor, a fall detection sensor, and the like) is transmitted to the server, and generative AI on the server side comprehensively analyzes these pieces of data. For example, it detects a situation such as “there is no utterance or movement for a certain period of time or more”, “a fall is detected”, “there is an abnormality in utterance content”, and “a heart rate and a body temperature show abnormal values” in real time, automatically notifies a family member or an administrative agency when an abnormality is recognized, and calls an API of an emergency transportation service to arrange an ambulance as necessary. For example, when no movement of an elderly person is detected at night, or when there is an utterance such as “help” or “painful”, an emergency response is immediately executed. Furthermore, the generative AI continuously learns the entire life of the elderly person, and automatically makes a proposal that contributes to an improvement in quality of life and a reduction in a feeling of loneliness, such as “propose a walk or exercise because a frequency of going out has decreased recently”, “provide nutritional guidance because a nutritional balance of meals is biased”, and “propose a hobby or an exchange event”. For example, it realizes detailed life support such as “propose an online exchange event because interaction with friends has decreased recently”, “encourage participation in a hobby activity”, and “regularly propose a video call with a family member”. Specifically, the present health condition detection unit adopts a multimodal neural network architecture. The present terminal converts a video frame acquired from a camera into an RGB image tensor. The present terminal converts 3-axis acceleration data acquired from an acceleration sensor into a time-series array. The present server executes a deep learning model equipped with a fusion layer that integrates these heterogeneous data. Input data to the present deep learning model is, for example, an image tensor with a resolution of 224×224. Input data to the present deep learning model is, for example, a 10-second acceleration time-series matrix acquired at a sampling rate of 50 Hz. Input data to the present deep learning model is, for example, a vital sign vector including numerical values of a heart rate and a blood pressure. The present deep learning model extracts a spatial feature map from an image tensor using a convolutional neural network. The present deep learning model extracts a temporal dependency from a time-series matrix using a long short-term memory network. The present deep learning model combines the extracted feature vectors and inputs them to a fully connected layer. The present deep learning model generates a probability score of binary classification indicating whether a current state is normal or abnormal as an output. The present deep learning model generates an output vector of a softmax function indicating a specific abnormality category such as a fall or loss of consciousness as an output. The present deep learning model outputs a health score from 0 to 100 indicating a comprehensive evaluation of a health condition as an output. A specific example of output data from the present deep learning model is, for example, a score of 0.98 indicating a fall probability. A specific example of output data from the present deep learning model is, for example, a classification label indicating a heart rate abnormality. A specific example of output data from the present deep learning model is, for example, a warning flag indicating a possibility of malnutrition. The present server generates an interrupt process when the output probability score exceeds a danger threshold. The present server immediately transmits a POST request including position information and vital data to an emergency transportation API. The present process realizes highly accurate situation recognition, which is impossible with a single sensor, by comprehensively analyzing a plurality of sensor data. The present process exhibits a technical effect of dramatically reducing false alarms. The present system is used in specific application fields such as a patient monitoring system in a smart hospital and a home medical support network. The present deep learning model is trained to minimize a cross-entropy error using a large amount of normal data and abnormal data. The present system artificially increases diversity of learning data using a data augmentation method. The present system prevents overfitting and improves generalization performance for an unknown situation. The present system provides a pure data-driven inference mechanism that does not depend on human observation.

In addition, since generative AI comprehensively analyzes a preference, a life rhythm, a health condition, and a past action history of each individual elderly person to realize personalized support, it is possible to flexibly respond in accordance with a situation, not limited to mere routine service provision. For example, a communication robot on the terminal side can propose “The weather is nice today, so why don't you go for a walk?”, remind “It is time for your medicine”, or notify “A message has arrived from your grandchild”. Furthermore, in cooperation with an administrative agency and a medical institution, social services such as “reservation for a regular health checkup”, “guidance on vaccination”, and “safety confirmation during a disaster” can also be provided in an integrated manner. Specifically, the present server executes a personalization engine based on a reinforcement learning algorithm. The present personalization engine constructs a user's action model using a Markov decision process. Input data to the present personalization engine is, for example, a context vector indicating a current state of a user. Input data to the present personalization engine is, for example, a history tensor indicating a user's acceptance or rejection of a past proposal. Input data to the present personalization engine is, for example, a structured JSON of weather data acquired from an external weather information API. The present personalization engine updates a state-action value function using a Q-learning algorithm. The present personalization engine generates an index of an optimal action to be executed next as an output. The present personalization engine generates a text character string of proposal content as an output. The present personalization engine outputs a schedule time stamp indicating a timing of a proposal as an output. A specific example of output data from the present personalization engine is, for example, an action ID indicating a “proposal of a walk”. A specific example of output data from the present personalization engine is, for example, text data for speech synthesis saying “It is time for your medicine”. A specific example of output data from the present personalization engine is, for example, an event flag that triggers transmission of vaccination guidance. The present server instantiates a corresponding service class based on the output action ID. The present server transmits the generated text data to the present terminal to output it as voice. The present process self-organizingly improves accuracy of a proposal by continuously receiving a user's reaction as feedback. The present process exhibits a technical effect of realizing high adaptability that is impossible with a uniform rule-based system. The present system is used in specific application fields such as a personalized medical support platform and a resident service portal of a smart city. The present system adopts an epsilon-greedy method to optimize a trade-off between exploration and exploitation. The present system uses a deep Q-network to process a high-dimensional state space. The present system updates a weight of a neural network using a mini-batch randomly sampled from an experience replay buffer. The present system ensures stability of learning. The present system is an improvement in computer technology that eliminates human intuition and rules of thumb and solves personalized support as a pure mathematical optimization problem.

For example, when generative AI on the server side analyzes an utterance, an action, and vital data of an elderly person acquired on the terminal side and detects a health abnormality or a disturbance in a life rhythm, it automatically notifies a family member or a medical institution, and automatically arranges emergency transportation or a medical consultation as necessary. For example, it can detect an abnormality such as “an elderly person falls and does not move for a certain period of time”, “a heart rate rises sharply”, and “meal and water intake significantly decreases” in real time, and immediately respond. Furthermore, it is also possible for a family member to remotely give an instruction such as “I want to request more cleaning this week” and “I want to reserve an accompaniment to a hospital visit next week”. Specifically, the present system adopts a cooperative inference architecture that dynamically divides an inference task between an edge device and a cloud server. The present terminal locally executes primary anomaly detection using a lightweight convolutional neural network. Input data to the lightweight model of the present terminal is, for example, a low-resolution grayscale image tensor acquired from a camera. Input data to the lightweight model of the present terminal is, for example, a short-time audio waveform vector acquired from a microphone. Input data to the lightweight model of the present terminal is, for example, a real-time heart rate stream from a wearable device. The lightweight model of the present terminal generates an initial probability score indicating a possibility of an abnormality as an output. A specific example of output data from the lightweight model of the present terminal is, for example, a score of 0.6 indicating a suspicion of a fall. The present terminal transmits high-resolution raw data to the present server only when the initial probability score exceeds a predefined threshold. The present server executes a secondary determination by applying a highly accurate transformer model with a large number of parameters to the received high-resolution data. Input data to the highly accurate model of the present server is, for example, a high-resolution RGB moving image tensor. Input data to the highly accurate model of the present server is, for example, a multivariate time-series matrix of detailed vital signs for the past 24 hours. The highly accurate model of the present server generates a final abnormality confirmation flag as an output. The highly accurate model of the present server outputs a classification label specifying a type of an abnormality as an output. The highly accurate model of the present server outputs a continuous value score from 0 to 10 indicating a degree of urgency as an output. A specific example of output data from the highly accurate model of the present server is, for example, a Boolean value of true indicating an abnormality confirmation. A specific example of output data from the highly accurate model of the present server is, for example, a category ID indicating a “sharp rise in heart rate”. A specific example of output data from the highly accurate model of the present server is, for example, numerical data indicating an urgency level of 9. The present server executes a routing protocol based on the output urgency score. The present server makes a direct API call to a system of a medical institution when the degree of urgency is high. The present server transmits an asynchronous message to a terminal of a family member when the degree of urgency is low. The present process dramatically reduces consumption of a network bandwidth. The present process exhibits a technical effect of maintaining extremely high detection accuracy while saving calculation resources of a cloud server. The present system is used in specific application fields such as a mission-critical remote medical monitoring system and an anomaly detection system for an industrial robot. The present system executes an autonomous control action with a latency in milliseconds without waiting for a determination by a human monitor. The present system provides a technical improvement of traffic optimization of a computer network and an improvement in distributed processing efficiency.

As described above, an embodiment of the present invention comprehensively realizes life support optimized for each individual elderly person, peace of mind for family members, and a solution to social issues while ensuring efficiency, scalability, and reliability of the entire system by intensively executing advanced analysis, determination, service linkage, data management, and settlement processing by generative AI on the server side, and taking charge of interaction with an elderly person, acquisition of sensor data, simple processing, and notification on the terminal side. Specifically, the present system manages each module on the server side as an independent microservice using a container orchestration tool. The present system evenly distributes requests from clients to a plurality of server nodes using a load balancer. The present system levels a traffic load of the entire system. The present system adopts a gRPC protocol for communication between respective microservices. The present system serializes data using protocol buffers. The present system compresses a data size and improves a communication speed compared to JSON-based REST communication. Input data to an orchestration engine in the present system is, for example, a metrics vector indicating a CPU usage rate and memory consumption of each server node. Input data to the orchestration engine in the present system is, for example, time-series data indicating a delay time of a network. Input data to the orchestration engine in the present system is, for example, an integer value indicating the number of currently active sessions. The present orchestration engine generates a command instructing activation of a new container instance as an output. The present orchestration engine outputs a configuration file for updating a traffic routing rule as an output. The present orchestration engine outputs a matrix indicating an optimization plan for resource allocation as an output. A specific example of output data from the present orchestration engine is, for example, a scaling command of “add three inference nodes”. A specific example of output data from the present orchestration engine is, for example, update data of a firewall rule that restricts access from a specific IP address band. A specific example of output data from the present orchestration engine is, for example, a configuration parameter for changing memory allocation from 2 GB to 4 GB. The present system applies the output command to a host operating system via a daemon process. The present process brings a downtime of the system close to zero even during a sudden concentration of access. The present process exhibits a technical effect of maximizing utilization efficiency of hardware resources. The present system is used in specific application fields such as a large-scale IoT platform and a global cloud service infrastructure. The present system provides a pure technical advance of an improvement in a resource management algorithm in a distributed computing environment, rather than mere automation of a business process. The present system includes a self-healing function that automatically executes failover when a failure occurs. The present system maintains availability of the system at 99.99 percent or more.

### System Configuration

The system according to the present embodiment comprises a communication unit, a service linkage unit, a remote monitoring unit, a settlement management unit, a health condition detection unit, a data management unit, a user interface unit, and a sensor linkage unit. The communication unit is implemented in a terminal such as a communication robot, a smart speaker, a tablet terminal, and a smartphone installed in a living space of an elderly person, acquires voice, video, an action, and a touch input of the elderly person using various interfaces such as a voice recognition microphone, a camera, a speaker, a touch panel, and a gesture sensor, and transmits these pieces of data to a server in real time. For example, when the elderly person utters “I have a headache today”, “I want to order milk and bread”, “Please clean the room”, “I want to make a video call with my grandchild”, “Tell me the news”, “Call an ambulance”, or the like, the communication unit acquires these utterances and actions with high accuracy and transmits them to generative AI on the server side. Furthermore, the communication unit also includes a function of temporarily buffering the acquired voice and video data in the terminal to prevent data loss even in the event of a communication failure, and a data synchronization function among a plurality of terminals. Specifically, the communication unit constructs a ring buffer data structure on a local memory of the terminal. The communication unit divides the acquired multimedia data into a certain chunk size and writes it into the ring buffer. The communication unit periodically polls a state of a network interface controller. The communication unit reads data from the buffer and adds it to a transmission queue only when a network connection is established. Input data to a data processing module in the communication unit is, for example, a multi-channel uncompressed PCM voice data array acquired from a microphone array. Input data to the data processing module in the communication unit is, for example, an uncompressed video tensor of 30 frames per second acquired from a camera. Input data to the data processing module in the communication unit is, for example, a two-dimensional coordinate vector indicating a capacitance change of a touch panel. The data processing module generates, as an output, a binary stream obtained by compressing voice data with an Opus codec. The data processing module outputs, as an output, a packet sequence obtained by compressing video data with an H.265 codec. The data processing module outputs, as an output, a JSON object in which a touch event is structured. A specific example of output data from the data processing module is, for example, a voice packet with a compression ratio of 1/10. A specific example of output data from the data processing module is, for example, a video stream composed of a key frame and a difference frame. A specific example of output data from the data processing module is, for example, touch event data including an X coordinate and a Y coordinate. The communication unit transmits the output compressed data to the server using a TCP or UDP protocol. The communication unit guarantees data ordering among a plurality of terminals using a timestamp-based logical clock algorithm. This process maintains data integrity even in an unstable network environment. This process produces a technical effect of preventing information loss due to packet loss. This system is used in specific application fields such as a communication infrastructure during a disaster and a data collection system in a mobile communication environment. This system executes acquisition of sensor data with low latency using hardware interrupt processing. This system manages a network socket at a kernel level of an operating system. This system is an improvement in computer technology that provides an autonomous data synchronization and error recovery mechanism that does not require human intervention.

The service linkage unit operates on the server side, and performs API linkage with various external services and IoT devices based on a request and a situation of the elderly person analyzed by generative AI. For example, in the case of shopping support, it calls an API of an EC site to acquire a product list, proposes it to the elderly person by a display or voice on the terminal side, and automatically executes an ordering process after an order is confirmed. In the case of meal support, it cooperates with an API of a nearby restaurant or a meal delivery service to collectively arrange from a menu proposal to ordering and delivery. In the case of a cleaning service, it calls an IoT control API of a robot vacuum cleaner to automatically operate it, or makes a reservation for a cleaning service by a person. In the case of medical support, it sets up a video call with a doctor using an API of a remote medical examination system, and arranges a pick-up and drop-off service of an autonomous vehicle as necessary. In the case of entertainment provision, it uses an API of a news distribution service or a music distribution service to realize functions such as “reading out the latest sports news”, “playing requested music”, and “acting as an agent for an event application”. The service linkage unit flexibly and seamlessly controls a series of flows of these service selections, proposals, and executions in accordance with an utterance or an action of the elderly person, and for example, when an utterance “I want to order milk and bread” is made, it also refers to a past purchase history and an inventory status, lists necessary products, confirms an order, and automatically adjusts a delivery date and time. Furthermore, the service linkage unit is also responsible for simultaneous use and scheduling of a plurality of services, acquisition of feedback on a service provision status, recording of a service usage history, and the like. Specifically, the service linkage unit expands a state machine based on a finite automaton on a memory to manage an execution state of each service. The service linkage unit processes a plurality of API calls in parallel using an asynchronous task queue. Input data to a scheduling algorithm in the service linkage unit is, for example, a numerical vector indicating an estimated time required for execution of each service. Input data to the scheduling algorithm in the service linkage unit is, for example, an adjacency matrix of a directed acyclic graph indicating a dependency relationship between services. Input data to the scheduling algorithm in the service linkage unit is, for example, a time window array indicating a desired time zone specified by a user. The scheduling algorithm generates, as an output, a schedule table including a start time and an end time of each task. The scheduling algorithm outputs, as an output, a lock acquisition order list for avoiding resource contention. The scheduling algorithm outputs, as an output, a weighting vector indicating a priority of a task. A specific example of output data from the scheduling algorithm is, for example, schedule data “cleaning robot operation start time 10:00”. A specific example of output data from the scheduling algorithm is, for example, a control flag “shopping API call priority high”. A specific example of output data from the scheduling algorithm is, for example, a state transition trigger of a state machine “waiting for medical reservation completion”. The service linkage unit sets a timer interrupt in accordance with the output schedule table and automatically executes a task. The service linkage unit exposes a webhook endpoint for receiving a callback from an external API. This process efficiently orchestrates a plurality of asynchronous processes without a deadlock. This process produces a technical effect of improving throughput of the entire system. This system is used in specific application fields such as robot control in a smart factory and a complex supply chain management system. This system controls a transmission frequency of requests using a token bucket algorithm in order to avoid an API rate limit. This system prevents network congestion. This system is a technical improvement that solves a multidimensional constraint satisfaction problem that surpasses human scheduling ability in real time by computing power of a computer.

The remote monitoring unit is mainly realized on the server side, and a dedicated application and a web interface are also provided to a terminal such as a smartphone and a personal computer used by a family member. The remote monitoring unit records and manages data such as an activity history, an order history, a health condition, and a service usage status of the elderly person in chronological order, and generative AI analyzes these pieces of data to detect an abnormal tendency and a change in a life rhythm. For example, it automatically extracts signs such as “there is no meal order for the past few days”, “an activity amount is significantly lower than usual”, and “words suggesting a poor physical condition are increasing in utterance content”, and transmits a push notification or an alert to the terminal of the family member. The family member can confirm “when and what a parent ordered”, “which service was used”, “a transition of a health condition”, “an emergency response history”, and the like in real time through the application or a web screen. Furthermore, it is also possible for a family member to remotely request an addition of a service, confirm a health condition, and give an emergency response instruction, and for example, an instruction or confirmation such as “I want to request more cleaning this week”, “I want to reserve an accompaniment to a hospital next week”, and “I want to confirm a recent health condition in detail” is possible. The remote monitoring unit also includes an automatic notification upon abnormality detection, a feedback reception from a family member, and a communication support function between a family member and an elderly person in order to ensure peace of mind for the family member. Specifically, the remote monitoring unit models a complex relationship between an action pattern of an elderly person and various events using a graph neural network. The server constructs graph structure data in which a node is an event and an edge is a temporal or causal relationship on a memory. Input data to the graph neural network is, for example, a node feature matrix indicating an action history of the past one week. Input data to the graph neural network is, for example, an adjacency matrix indicating a transition probability between events. Input data to the graph neural network is, for example, a timestamp vector indicating an occurrence time of each event. The graph neural network aggregates feature information between adjacent nodes using a message passing algorithm. The graph neural network generates, as an output, a single scalar value indicating an abnormality degree of the entire graph. The graph neural network outputs, as an output, a distribution of attention scores specifying a specific node causing an abnormality. The graph neural network outputs, as an output, a probability distribution vector predicting a future action. A specific example of output data from the graph neural network is, for example, an abnormality degree score of 0.89 for the entire graph. A specific example of output data from the graph neural network is, for example, a high attention weight for a “meal not ordered” node. A specific example of output data from the graph neural network is, for example, a probability of 0.75 predicting “a decrease in an activity amount on the next day”. The server activates an alert generation module when the output abnormality degree score exceeds a specified threshold. The server generates a natural language text explaining a root cause of the abnormality based on the attention score. The server transmits a push notification including the generated text to the terminal of the family member. This process detects an abnormality due to a combination of a plurality of minute changes that would be missed by monitoring a single piece of time-series data with high accuracy. This process produces a technical effect of significantly reducing a false positive rate. This system is used in specific application fields such as an intrusion detection system in cybersecurity and a fraud detection system for financial transactions. This system receives a request from the terminal of the family member via a RESTful API and asynchronously updates a record in a database. This system processes simultaneous accesses from a plurality of users while maintaining data consistency. This system is a technical improvement that realizes correlation analysis of multidimensional data exceeding human cognitive limits by parallel processing capability of a computer.

The settlement management unit is realized on the server side, centrally aggregates usage fees of all services provided through the service linkage unit, and performs a collective settlement process in cooperation with an API of a cashless payment service. For example, even when a plurality of services such as shopping, a meal, cleaning, medical support, and entertainment are used, it automatically summarizes usage details and performs automatic withdrawal from a settlement account of an elderly person or a family member at the end of a month or every time of use. A settlement status and usage details can be confirmed by a family member or an elderly person from a terminal at any time. For example, it can immediately respond to a request such as “how much did food expenses cost this month”, “I want to confirm usage details of a medical service”, and “I want to confirm a payment status of a cleaning service”. The settlement management unit also has functions such as a re-billing process at the time of a settlement error, automatic transmission of usage details, a payment notification to a family member, and a usage upper limit setting. Specifically, the settlement management unit atomically processes billing requests from a plurality of service modules using a distributed transaction processing system. The settlement management unit executes a two-phase commit protocol to guarantee data consistency in all nodes. Input data to a billing aggregation algorithm in the settlement management unit is, for example, a record array of microtransactions transmitted from each service. Input data to the billing aggregation algorithm in the settlement management unit is, for example, a profile vector indicating a discount rate or a subscription plan for each user. Input data to the billing aggregation algorithm in the settlement management unit is, for example, an external parameter matrix indicating a fluctuation in an exchange rate or a tax rate. The billing aggregation algorithm generates, as an output, a floating-point number indicating a final billing amount for each user. The billing aggregation algorithm outputs, as an output, a hierarchical JSON tree structure indicating a usage breakdown of each service. The billing aggregation algorithm outputs, as an output, an encrypted payload to be transmitted to a settlement gateway. A specific example of output data from the billing aggregation algorithm is, for example, numerical data of a total billing amount of 15,000 yen. A specific example of output data from the billing aggregation algorithm is, for example, breakdown data of food expenses of 5,000 yen and medical expenses of 10,000 yen. A specific example of output data from the billing aggregation algorithm is, for example, a settlement token encrypted by an RSA algorithm. The settlement management unit transmits the output encrypted payload to an external settlement server using a TLS protocol. The settlement management unit analyzes a response code from the settlement server, and adds a task to a retry queue with exponential backoff when an error occurs. This process completely prevents omission or duplication of a settlement process even at the time of a momentary network interruption or an overload of an external server. This process produces a technical effect of maximizing reliability and availability of the system. This system is used in specific application fields such as a large-scale E-commerce platform and a pay-as-you-go billing system of a cloud service. This system adds a digital signature for detecting data falsification to each transaction using a public key infrastructure. This system optimizes an index structure of a database to respond to a search query for millions of usage details in milliseconds. This system is a technical improvement that eliminates an aggregation error due to human manual work and realizes a secure value transfer via a computer network.

The health condition detection unit is realized on both the terminal side and the server side. On the terminal side, data acquired from a microphone, a camera, and various sensors (such as an acceleration sensor, a heart rate sensor, a temperature sensor, a blood pressure sensor, a sleep sensor, and a fall detection sensor) is transmitted to the server, and generative AI on the server side comprehensively analyzes these pieces of data. For example, it detects a situation such as “there is no utterance or movement for a certain period of time or more”, “a fall is detected”, “there is an abnormality in utterance content”, and “a heart rate or a body temperature indicates an abnormal value” in real time, automatically notifies a family member or an administrative agency when an abnormality is recognized, and calls an API of an emergency transportation service to arrange an ambulance as necessary. The health condition detection unit has various functions such as accumulation and analysis of daily health data, early discovery of a health abnormality, trend analysis of a health condition, automatic proposal of health advice, and automatic response in an emergency. Specifically, the health condition detection unit executes a deep autoencoder network for abnormality detection on the server side. The terminal digitally samples an analog signal acquired from various sensors by a built-in microcontroller and applies a noise removal filter. Input data to the autoencoder network is, for example, a multivariate time-series tensor acquired synchronously from a plurality of sensors. Input data to the autoencoder network is, for example, a matrix indicating a time-series transition of skeleton coordinates extracted from a camera video. Input data to the autoencoder network is, for example, a spectrogram image of an acoustic feature amount extracted from a voice signal. The autoencoder network comprises an encoder layer that compresses input data into a low-dimensional latent space vector. The autoencoder network comprises a decoder layer that restores original input data from a latent space vector. The autoencoder network generates, as an output, a restored multivariate time-series tensor. The autoencoder network outputs, as an output, a reconstruction error vector between input data and restored data. The autoencoder network outputs, as an output, an abnormality probability score based on a reconstruction error. A specific example of output data from the autoencoder network is, for example, a reconstruction error value of 2.5 for heart rate data. A specific example of output data from the autoencoder network is, for example, an abnormality probability score of 0.95 indicating a rapid change in skeleton coordinates. A specific example of output data from the autoencoder network is, for example, an abnormality probability score of 0.05 indicating a normal state. The server emulates a hardware interrupt and executes an emergency response process with the highest priority when the output abnormality probability score exceeds a dynamically calculated statistical threshold. The server generates a JSON payload including GPS coordinate data in the emergency response process and transmits it to an emergency transportation API. This process makes it possible to perform detection with high sensitivity even for an unknown abnormality pattern. This process produces a technical effect of capturing a complex sudden change in a health condition that cannot be handled by a rule-based system. This system is used in specific application fields such as a patient monitoring system in an intensive care unit and a condition management system for a professional athlete. This system solves a problem of a shortage of labeled abnormal data by pre-training an autoencoder using only normal health data. This system executes online learning that continuously fine-tunes a weight of a network using a gradient descent method. This system adapts to an individual's secular change. This system provides a technical improvement of continuous and multidimensional data analysis by a computer as well as assisting a diagnosis by a human doctor.

The data management unit is realized on the server side, and safely and efficiently records and manages all data (such as utterance data, video data, sensor data, a service usage history, health data, settlement data, and a communication history with a family member) generated in the entire system in chronological order. The data management unit has functions such as data encryption, access authority management, backup, data erasure, and provision of an interface for data analysis, and ensures privacy protection and security. For example, it performs appropriate authentication and access control in response to a data viewing request from a family member or a medical institution. Specifically, the data management unit constructs a hybrid data storage architecture combining a relational database and a NoSQL database. The data management unit stores structured settlement data and a user profile in a relational database to guarantee ACID properties. The data management unit saves a voice file and a video stream, which are unstructured data, in an object storage. Input data to a data writing process in the data management unit is, for example, a log record in a JSON format asynchronously transmitted from each module. Input data to the data writing process in the data management unit is, for example, a media file in a binary format. Input data to the data writing process in the data management unit is, for example, a metadata vector including an IP address of an access source and a user agent. The data management unit generates, as an output, a commit response indicating a successful writing to a database. The data management unit outputs, as an output, a unique access URI to saved data. The data management unit outputs, as an output, a checksum for falsification prevention including a hash value of data. A specific example of output data from the data management unit is, for example, a status code indicating transaction completion. A specific example of output data from the data management unit is, for example, a file path character string on an object storage. A specific example of output data from the data management unit is, for example, a hash character string calculated by a SHA256 algorithm. The data management unit indexes and saves the output URI in a metadata table. The data management unit shortens an execution time of a search query for hundreds of millions of records to a logarithmic time order using a B-tree index structure. This process executes reading and writing of petabyte-class large-scale data with extremely low latency. This process produces a technical effect of eliminating an IO bottleneck of the system. This system is used in specific application fields such as a large-scale electronic medical record system and a national-level health information infrastructure. This system encrypts both saved data and data in communication using an advanced encryption standard algorithm. This system caches a role-based access control list on a memory to speed up an authentication process. This system executes periodic snapshot acquisition and asynchronous replication to a remote location to ensure disaster recovery. This system is a technical improvement that optimizes a storage hierarchy and a search algorithm of a computer, rather than a mere data repository.

The user interface unit is implemented on the terminal side, and provides various interfaces such as voice, video, touch, gesture, and text so that an elderly person and a family member can intuitively use the system. For example, when an elderly person utters “Tell me the news”, it reads out the latest news by voice and displays a detailed article on a touch panel. When a family member operates “I want to confirm a health condition of a parent” from a smartphone, it displays health data and a service usage history in a graph or a list. The user interface unit learns an operation history and a preference of a user, and automatically generates an individually optimized screen configuration and proposal. Specifically, the user interface unit executes high-speed screen drawing by hardware acceleration using a graphics processing unit of the terminal. The user interface unit constructs a virtual DOM tree on a memory and differentially updates only a component having a state change. Input data to a rendering engine in the user interface unit is, for example, a JSON object including the latest health data received from the server. Input data to the rendering engine in the user interface unit is, for example, a gaze point coordinate vector acquired from a line-of-sight tracking sensor of a user. Input data to the rendering engine in the user interface unit is, for example, illuminance data acquired from an ambient light sensor. The rendering engine generates, as an output, a pixel color array to be transmitted to a display. The rendering engine outputs, as an output, PCM waveform data to be transmitted to a voice synthesis chip. The rendering engine outputs, as an output, a PWM signal for driving a tactile feedback actuator. A specific example of output data from the rendering engine is, for example, RGB frame buffer data with a resolution of 1920×1080. A specific example of output data from the rendering engine is, for example, voice waveform data “Here is the latest news”. A specific example of output data from the rendering engine is, for example, a control signal indicating a vibration pattern when a button is pressed. The user interface unit transfers the output pixel data to a display controller to update a screen. The user interface unit locally executes a reinforcement learning agent and optimizes a parameter of a screen layout using a time until completion of a user operation as a reward. This process realizes dynamic interface reconfiguration that minimizes a cognitive load of a user. This process produces a technical effect of reducing an operation error rate and improving accessibility of the system. This system is used in specific application fields such as a cockpit display of an aircraft and a human-machine interface of an industrial control panel. This system automatically adjusts a backlight luminance and a contrast ratio of a display based on ambient light data. This system dynamically enlarges a font size of an area focused by a user based on a line-of-sight coordinate vector. This system is a technical improvement in which a computer autonomously constructs an optimal input/output environment by eliminating the need for human manual setting.

The sensor linkage unit is implemented on the terminal side, acquires data from various sensors (such as an acceleration sensor, a heart rate sensor, a temperature sensor, a blood pressure sensor, a sleep sensor, a fall detection sensor, a door opening/closing sensor, and an illuminance sensor) in real time, and transmits it to the data management unit and the health condition detection unit. For example, when a fall detection sensor detects a rapid acceleration change, it immediately notifies the server, and generative AI determines urgency. When a sleep sensor detects a lack of sleep, it proposes an improvement in a life rhythm. When a door opening/closing sensor has not been opened or closed for a long time, it warns of a decrease in an activity amount. When an illuminance sensor detects abnormal brightness at night, it notifies a possibility of night wandering. Specifically, the sensor linkage unit executes data acquisition from a sensor with microsecond accuracy using a hardware interrupt function of a microcontroller. The sensor linkage unit performs serial communication with a plurality of sensor modules using an I2C or SPI communication protocol. Input data to a data preprocessing module in the sensor linkage unit is, for example, an analog voltage signal output from an acceleration sensor. Input data to the data preprocessing module in the sensor linkage unit is, for example, a digital high-low signal from a door opening/closing sensor. Input data to the data preprocessing module in the sensor linkage unit is, for example, raw waveform data of a photoplethysmogram from a heart rate sensor. The data preprocessing module converts a voltage signal into a discrete digital value using an analog-to-digital converter. The data preprocessing module applies a Kalman filter to reduce sensor noise and estimate a true signal value. The data preprocessing module generates, as an output, a floating-point vector of 3-axis acceleration from which noise has been removed. The data preprocessing module outputs, as an output, a Boolean value indicating an opening/closing state of a door. The data preprocessing module outputs, as an output, a numerical array in milliseconds indicating a peak interval of a heart rate. A specific example of output data from the data preprocessing module is, for example, vector data indicating acceleration values of an X-axis, a Y-axis, and a Z-axis. A specific example of output data from the data preprocessing module is, for example, a true Boolean value indicating that a door is opened. A specific example of output data from the data preprocessing module is, for example, numerical data of a heartbeat interval of 800 milliseconds. The sensor linkage unit writes the output data into a local ring buffer together with a timestamp. The sensor linkage unit activates a direct memory access controller to transfer data to a network chip at high speed when data in the buffer reaches a certain amount or exceeds a specific threshold. This process efficiently processes a large amount of sensor data while minimizing CPU intervention. This process produces a technical effect of reducing power consumption of a terminal and extending a battery driving time. This system is used in specific application fields such as an environment recognition system of an autonomous vehicle and a predictive maintenance system of a factory. This system holds a calibration parameter of a sensor in a non-volatile memory and corrects a drift of the sensor due to a temperature change by software. This system provides an improvement in computer technology of advanced signal processing and resource management in an edge device, rather than mere data collection.

Specific examples of a prompt sentence to be read by generative AI necessary for implementing the present invention include “Analyze utterance content of an elderly person and determine a request, a physical condition change, and urgency”, “Propose an optimal life support service based on a past service usage history and health data”, “Detect a health abnormality, a fall, and a decrease in an activity amount from sensor data, and notify a family member or an administrative agency as necessary”, “Extract appropriate information in response to a remote instruction or an inquiry from a family member, and answer in an easy-to-understand manner”, “Learn a preference and a life rhythm of an elderly person, and generate personalized entertainment and health advice”, and “Integrate a plurality of service usage statuses and settlement data, and automatically summarize and present usage details and a payment status”. Specifically, the server executes a prompt engineering module to dynamically construct an input text to a large language model. The prompt engineering module combines a system prompt, a user context, and a task instruction to generate one long token sequence. Input data to the prompt engineering module is, for example, a statistical value vector of vital data for the past three days retrieved from a database. Input data to the prompt engineering module is, for example, an environment variable tensor indicating a current time and weather. Input data to the prompt engineering module is, for example, the latest utterance text character string of an elderly person. The prompt engineering module generates, as an output, a prompt character string optimized for a context window size of a large language model. The prompt engineering module outputs, as an output, a format instruction text for forcing an output format of a model to JSON. The prompt engineering module outputs, as an output, a pair array of specific examples for few-shot learning. A specific example of output data from the prompt engineering module is, for example, an instruction character string “Output urgency from 0 to 1 based on the following vital data”. A specific example of output data from the prompt engineering module is, for example, a format constraint character string “Set an output format to JSON having intent as a key”. A specific example of output data from the prompt engineering module is, for example, a text block including a past similar case and its correct label. The server inputs the output prompt character string to a tokenizer and converts it into an integer index array. The server supplies the index array to an input layer of a large language model to start an inference process. This process suppresses hallucination of a large language model and ensures accuracy and consistency of an output. This process produces a technical effect of efficiently executing a complex inference task with a single model call. This system is used in specific application fields such as an automatic programming support tool and an advanced legal document analysis system. This system monitors a length of a prompt and executes a sliding window algorithm that automatically summarizes or deletes a past context with low importance when a token limit is exceeded. This system provides a technical improvement of automating a human prompt creation work and optimizing a natural language processing pipeline by a computer.

In this way, the system according to the present embodiment is configured such that each unit closely cooperates to support the entire life of an elderly person in a multifaceted and individually optimized manner with generative AI as a core, thereby realizing peace of mind and efficiency for a family member and society as a whole. Specifically, this system adopts an event-driven architecture to make a degree of coupling between modules loose. This system executes asynchronous communication by a publish-subscribe model via a message broker. Input data to a message routing engine in this system is, for example, an event message including a topic name issued from the sensor linkage unit. Input data to the message routing engine in this system is, for example, a payload of an emergency alert issued from the health condition detection unit. Input data to the message routing engine in this system is, for example, notification data of transaction completion issued from the settlement management unit. The message routing engine executes, as an output, a routing process of distributing a message to a reception queue of an appropriate subscriber. The message routing engine generates, as an output, an acknowledgment signal corresponding to a delivery guarantee level of a message. The message routing engine outputs, as an output, a control command for moving a message that has failed to be processed to a dead letter queue. A specific example of output data from the message routing engine is, for example, routing path information addressed to a specific microservice. A specific example of output data from the message routing engine is, for example, an ACK packet indicating delivery completion. A specific example of output data from the message routing engine is, for example, an error code instructing a retransmission process. This system dynamically updates a forwarding table of a network switch based on the output routing information. This process realizes fault tolerance that continues operations of other modules even when a failure occurs in a part of the system. This process produces a technical effect of dramatically improving scalability of the entire system. This system is used in specific application fields such as a large-scale financial transaction system and a global logistics tracking system. This system continuously collects a CPU usage rate and a memory consumption amount of each module by a monitoring agent. This system automatically specifies a resource bottleneck based on the collected metrics and executes container relocation. This system provides a fundamental improvement in computer technology of optimizing resource management and communication control in a distributed system, rather than a mere collection of services.

### Implementation Steps

### Step 1: Start of Elderly Person Interaction

An elderly person transmits a request, a question, a complaint of a physical condition, a daily conversation, a service request, and the like to a terminal such as a communication robot, a smart speaker, and a tablet terminal installed in a living space by a method such as voice, touch, gesture, and video. For example, various utterances and operations such as “I want to order milk and bread”, “Please clean”, “I have a headache today”, “I want to talk to my grandchild”, “Tell me the news”, and “Call an ambulance” are included. The terminal acquires these inputs in real time and transmits them to a server. Specifically, the terminal makes an event listener process operating at a kernel level of an operating system resident. The terminal activates a voice activity detection algorithm when a voice input level from a microphone array exceeds a set noise gate threshold. Input data to an input processing module in the terminal is, for example, a multi-channel raw voice waveform tensor acquired at a sampling rate of 48 kHz. Input data to the input processing module in the terminal is, for example, a multi-touch coordinate matrix from a capacitive touch panel. Input data to the input processing module in the terminal is, for example, three-dimensional point cloud data acquired from a depth camera. The input processing module generates a feature vector of Mel-frequency cepstral coefficients extracted from a voice waveform as an output. The input processing module outputs a classification ID of a gesture recognized from a touch trajectory as an output. The input processing module outputs a joint coordinate vector of a skeleton estimated from point cloud data as an output. A specific example of output data from the input processing module is, for example, a 128-dimensional floating-point vector indicating a voice feature. A specific example of output data from the input processing module is, for example, a gesture ID indicating “swipe right”. A specific example of output data from the input processing module is, for example, XYZ numerical data indicating spatial coordinates of a right wrist. The terminal serializes the output feature data into a protocol buffer format. The terminal transmits the serialized data to an API gateway of the server via a TLS encrypted tunnel. This process filters unnecessary environmental noise and an erroneous operation on an edge side. This process has a technical effect of reducing an amount of data transmitted to the server and saving a network bandwidth. The system is used in specific application fields such as an integrated controller of a smart home and a hands-free operation terminal in a factory. The terminal synchronizes a plurality of input modalities with a time stamp. The terminal packages simultaneous inputs of voice and gesture as one composite event. The system provides an improvement in input interface technology that converts a human intention into structured data processable by a computer.

### Step 2: Utterance Analysis and Intention Extraction by Generative AI

On the server side, generative AI receives voice and text data transmitted from an elderly person, and performs multi-layered analysis such as semantic analysis of utterance content, emotion analysis, urgency determination, health condition estimation, and life pattern extraction by natural language processing. For example, a sign of a health abnormality is extracted from an utterance “I have a headache today”, and a shopping request is extracted from an utterance “I want to order milk and bread”. Specific examples of a prompt sentence to be read by the generative AI include “Analyze utterance content of an elderly person and determine a request, a physical condition change, and urgency”, “Extract a service request and a health condition from utterance content”, and the like. Specifically, the server deploys a voice recognition module and a large language model on a memory. The voice recognition module receives voice waveform data transmitted from the terminal. The voice recognition module converts the voice waveform data into a frequency-domain spectrogram by fast Fourier transform. The voice recognition module inputs the spectrogram to a convolutional neural network. The voice recognition module generates text character string data as an output. The server tokenizes the text character string data by a morphological analyzer. The server inputs the tokenized one-dimensional array data to the large language model. The large language model is composed of a transformer architecture having tens of billions of parameters. Input data to the large language model includes, for example, a token array of a text “I have a headache today” and a token array of a system prompt. The number of dimensions of the input data to the large language model is, for example, a 4096-dimensional embedding vector. The large language model calculates a dependency relationship between tokens using a self-attention mechanism. The large language model generates structured data including an intention classification label, an emotion score, an urgency score, and an extracted entity as an output. A specific example of output data of the large language model is a key-value type data structure in which an intention classification label is “health consultation”, an emotion score is minus 0.8, an urgency score is 0.7, and an entity is “headache”. The server receives the output data. The server determines whether or not the urgency score exceeds 0.5, which is a preset threshold. When the urgency score exceeds the threshold, the server immediately transfers the structured data to a subsequent emergency response module. When the urgency score is equal to or less than the threshold, the server transfers the data to a normal service proposal module. By this process, the system determines an intention and urgency of an utterance with high accuracy and low latency in milliseconds without intervention of a human operator. The system thereby has a technical effect of preventing a delay in response in an emergency. Specific application fields of the system are a smart home system for an elderly person and a remote medical support system. The large language model executes self-supervised learning using a medical corpus and a daily conversation corpus in pre-training. The large language model updates a weight parameter by a gradient descent method so as to minimize a cross-entropy error function using an intention classification dataset annotated by an expert in fine-tuning. The system thereby dramatically improves intention extraction accuracy in a specific domain. The system adopts a non-conventional processing method based on semantic similarity in a high-dimensional vector space instead of simple keyword matching. The system thereby realizes robust analysis even for orthographical variants and ambiguous expressions.

### Step 3: Service Proposal and Optimization Process

Generative AI refers to a past utterance history, a service usage history, health data, and preference information of an elderly person, and generates an individually optimized service proposal and countermeasure. For example, it makes a flexible proposal according to a situation, such as “propose a fish dish because orders for fish dishes have decreased recently”, “automatically propose cleaning because there is a tendency to desire cleaning every Monday”, and “propose medical support with priority because utterances of poor physical condition are increasing”. Specific examples of a prompt sentence to be read by the generative AI include “Propose an optimal life support service based on a past service usage history and health data”, “Learn a preference and a life rhythm, and generate a personalized proposal”, and the like. Specifically, the server constructs a deep learning model functioning as a recommendation system. The deep learning model has an architecture combining a multilayer perceptron and a self-attention mechanism. The server acquires a past utterance history, a service usage history, health data, and preference information of an elderly person from a database. The server normalizes these heterogeneous data and applies an embedding layer for mapping them to the same latent space. Input data to the deep learning model includes, for example, a multidimensional tensor representing a service usage history for the past 30 days, a chronological array of vital signs for the latest one week, and a categorical vector indicating a user attribute. The number of dimensions of the input data to the deep learning model is, for example, a 256-dimensional combined vector. The deep learning model repeatedly applies a non-linear transformation to the input combined vector. The deep learning model generates a probability distribution of a recommendation score for each providable service as an output. A specific example of output data of the deep learning model is a numerical vector in which a recommendation score of a fish dish delivery service is 0.85, a recommendation score of a cleaning agency service is 0.92, and a recommendation score of a medical consultation service is 0.78. The server receives the output data. The server extracts the top three services having the highest recommendation scores. The server transfers the extracted service information to a subsequent user interface generation module. The user interface generation module generates a drawing command for displaying the service information on a display of the terminal. By this process, the system predicts a potential need from a huge amount of history data with speed and accuracy exceeding human cognitive ability. The system thereby has a technical effect of improving a quality of life of a user and maximizing a service usage rate. Specific application fields of the system are a personalized life support platform and a resident service provision system in a smart city. The deep learning model uses an acceptance history of a user for a past proposal as a correct label during learning. The deep learning model updates a weight parameter by backpropagation so as to minimize a binary cross-entropy loss function. The system adopts a non-conventional processing method of learning an interaction of feature amounts in a high-dimensional space instead of rule-based conditional branching. The system thereby realizes an adaptive proposal even for an unknown situation and a complex change in a life pattern.

### Step 4: External Service and IoT Device Linkage

A service linkage unit performs API linkage with an external service and an IoT device, such as an EC site, a meal delivery service, a cleaning service, a remote medical examination system, a pick-up and drop-off service, a news distribution service, a music distribution service, a robot vacuum cleaner, and a health management IoT device, based on an analysis result of generative AI, and automatically executes ordering, delivery, cleaning, remote medical examination, pick-up and drop-off, music playback, news provision, event application, and the like. For example, when an utterance “I want to order milk and bread” is made, it calls an API of an EC site to order products and adjusts a delivery date and time. Specifically, the service linkage unit includes an API gateway based on a microservice architecture. The service linkage unit receives structured data output from the generative AI. The service linkage unit analyzes an intention classification label and an extracted entity included in the structured data. The service linkage unit dynamically determines an API endpoint of an external service to be linked based on an analysis result. Input data to the service linkage unit is, for example, a key-value type data structure including a service identifier, a user identifier, a list of order items, and a desired delivery date and time. A specific example of input data to the service linkage unit is character string data in which a service identifier is “EC site A”, order items are “milk, bread”, and a desired delivery date and time is “3:00 PM today”. The service linkage unit converts the input data into a specific data format required by an external API. The service linkage unit transmits the converted data to an external server as an HTTP request. The service linkage unit receives response data including an execution result status and a transaction ID as an output from the external server. A specific example of output data of the service linkage unit is numerical and character string data in which a status code is 200, a transaction ID is “TXN12345”, and a scheduled delivery time is “3:15 PM”. The service linkage unit receives the response data. When the status code indicates normal, the service linkage unit transfers a transaction completion message to a subsequent notification module. The notification module inputs the message to a voice synthesis engine, and causes a speaker of a terminal to play a completion notification. By this process, the system abstracts communication with external systems having a plurality of different protocols, and realizes seamless linkage. The system thereby has a technical effect of reducing an operation burden on a user to zero and ensuring certainty of task execution. Specific application fields of the system are a hub device of a smart home and an integrated concierge service. The service linkage unit automatically executes a retry process using an exponential backoff algorithm when a communication error or a timeout occurs. The system adopts a non-conventional processing method that eliminates a re-input operation by a human manual operation and improves reliability of a computer network. The system thereby exhibits high fault tolerance even against a temporary network failure.

### Step 5: Constant Monitoring of Health Condition and Activity Status

Data acquired from various sensors (an acceleration sensor, a heart rate sensor, a temperature sensor, a blood pressure sensor, a sleep sensor, a fall detection sensor, a door opening/closing sensor, an illuminance sensor, and the like) on a terminal side is transmitted to a server, and generative AI comprehensively analyzes these pieces of data. For example, a situation such as “there is no utterance or movement for a certain period of time or more”, “a fall is detected”, and “a heart rate and a body temperature show abnormal values” is detected in real time, and when an abnormality is recognized, a family member or an administrative agency is automatically notified, and an API of an emergency transportation service is called to arrange an ambulance as necessary. Specific examples of a prompt sentence to be read by the generative AI include “Detect a health abnormality, a fall, and an activity amount decrease from sensor data, and notify a family member or an administrative agency as necessary”, and the like. Specifically, the server constructs a hybrid neural network combining a long short-term memory network specialized for time-series data analysis and a transformer model. The server receives raw data sampled in milliseconds from various sensors on the terminal side. The server applies noise removal filtering and a normalization process to the raw data. Input data to the hybrid neural network includes, for example, a chronological array of a heart rate for the past 24 hours, continuous waveform data of a three-axis acceleration sensor, and a numerical vector of indoor temperature and humidity. The number of dimensions of the input data to the hybrid neural network is, for example, a 128-dimensional time-series tensor. The hybrid neural network extracts a temporal dependency relationship and a spatial correlation relationship from the input time-series tensor. The hybrid neural network generates a normal probability score of a current health condition, a fall occurrence probability score, and a warning flag of an activity amount decrease as an output. A specific example of output data of the hybrid neural network is numerical and logical value data in which a normal probability score is 0.1, a fall occurrence probability score is 0.95, and a warning flag is true. The server receives the output data. The server determines whether or not the fall occurrence probability score exceeds 0.8, which is a preset threshold. When the fall occurrence probability score exceeds the threshold, the server immediately transmits a trigger signal to a subsequent emergency notification module. The emergency notification module receives the trigger signal, and executes a push notification to a smartphone of a family member and an API call of an emergency transportation service in parallel. By this process, the system detects a minute change in vital signs and a sudden accident in real time, which are impossible by human visual monitoring. The system thereby has an extremely important technical effect of improving a lifesaving rate and preventing aggravation. Specific application fields of the system are an elderly person monitoring system and a patient monitoring system in a hospital. The hybrid neural network uses a contrastive learning method that contrasts data of a normal life pattern with data at the time of occurrence of an abnormality during learning. The hybrid neural network optimizes a margin ranking loss function in order to maximize accuracy of abnormality detection. The system adopts a non-conventional processing method of dynamic abnormality detection in a high-dimensional feature space instead of simple threshold determination. The system thereby realizes highly accurate monitoring without a false alarm tailored to an individual life rhythm.

### Step 6: Remote Monitoring and Information Provision to Family Member

A remote monitoring unit records and manages data such as an activity history, an order history, a health condition, and a service usage status of an elderly person in chronological order, and provides information in real time to a terminal such as a smartphone and a personal computer used by a family member through a dedicated application and a web interface. For example, a family member can confirm “when and what a parent ordered”, “which service was used”, “a transition of a health condition”, “an emergency response history”, and the like. When an abnormality is detected, a push notification and an alert are automatically transmitted to a family member. Specifically, the remote monitoring unit includes a distributed database and a real-time data streaming engine. The remote monitoring unit receives log data of an activity history, an order history, a health condition, and a service usage status transmitted asynchronously from each subsystem. The remote monitoring unit adds a time stamp and a user identifier to the log data and stores it in the distributed database. The remote monitoring unit executes a data aggregation process in response to a request from a terminal of a family member. Input data to the remote monitoring unit is, for example, a query vector including a specific user identifier, a search start date and time, and a search end date and time. A specific example of input data to the remote monitoring unit is character string data in which a user identifier is “USER987”, a search start date and time is “Oct. 1, 2023”, and a search end date and time is “Oct. 31, 2023”. The remote monitoring unit extracts a corresponding record from the distributed database at high speed based on the query vector. The remote monitoring unit statistically processes the extracted record, and generates structured data for graph drawing as an output. A specific example of output data of the remote monitoring unit is a dataset including a one-dimensional array indicating a daily step count transition, key-value data for a pie chart indicating a service usage ratio, and a list of abnormality detection events. The remote monitoring unit transmits the output data to the terminal of the family member. The terminal of the family member receives the output data, and renders it as a visual dashboard on a screen of the dedicated application. By this process, the system instantly converts a huge amount of unstructured log data into a format intuitively understandable by a human. The system thereby has a technical effect of eliminating anxiety of a family member living apart and presenting an appropriate intervention timing. Specific application fields of the system are a nursing care support platform and a personal health record management system. When receiving an alert signal from an abnormality detection module, the remote monitoring unit ignores a normal polling interval and immediately establishes WebSocket communication. The remote monitoring unit transmits a forced push notification to the terminal of the family member. The system adopts a non-conventional processing method that guarantees immediate transmission of information by an event-driven architecture without depending on a regular confirmation operation by a human. The system thereby eliminates information asymmetry and supports quick decision-making.

### Step 7: Settlement Process and Usage Details Management

A settlement management unit centrally aggregates usage fees of all services provided through a service linkage unit, and performs a collective settlement process in cooperation with an API of a cashless payment service. Usage details and a settlement status can be confirmed at any time by a family member or an elderly person from a terminal. For example, it can immediately respond to a request such as “how much did food expenses cost this month” and “I want to confirm usage details of a medical service”. Specifically, the settlement management unit includes a transaction processing engine and an encryption module. The settlement management unit receives billing data together with a usage completion notification of each service from the service linkage unit. The settlement management unit records the billing data in a virtual ledger database for each user. The settlement management unit activates a collective settlement batch process at a preset closing date or when a usage limit is reached. Input data to the settlement management unit is, for example, structured data including a user identifier, a list of all transaction records within a target period, and a total billing amount. A specific example of input data to the settlement management unit is numerical and character string data in which a user identifier is “USER987”, the number of transactions is 15, and a total billing amount is “54,320 yen”. The settlement management unit converts the input data into a secure payload format required by an API of a cashless payment service. The settlement management unit encrypts a payload by an Advanced Encryption Standard algorithm using the encryption module. The settlement management unit transmits the encrypted payload to an external settlement gateway. The settlement management unit receives response data including a settlement completion flag, an approval number, and a time stamp as an output from the external settlement gateway. A specific example of output data of the settlement management unit is data in which a settlement completion flag is true, an approval number is “AUTH12345”, and a time stamp is “11:59 PM on Oct. 31, 2023”. The settlement management unit receives the response data. When the settlement completion flag is true, the settlement management unit updates a status of a corresponding record in the virtual ledger database to “settled”. The settlement management unit shifts a process to a subsequent details generation module. The details generation module generates a usage details report from the updated database, and provides it to a terminal of a user in a viewable format. By this process, the system integrally manages complex microtransactions spanning a plurality of providers with a single interface. The system thereby has a technical effect of dramatically reducing a payment management burden on a user and minimizing an unpaid risk. Specific application fields of the system are an integrated subscription management platform and a common settlement infrastructure of a smart city. The settlement management unit adopts a two-phase commit protocol in a distributed transaction, and completely prevents data inconsistency. The system adopts a non-conventional processing method that eliminates a manual aggregation and invoice issuing process by a human and is completed by secure automatic communication between computers. The system thereby maximizes transparency and safety of a financial transaction.

### Step 8: Feedback, Learning, and Continuous Optimization

Generative AI continuously learns an entire life of an elderly person, comprehensively analyzes a service usage history, a health condition, feedback from a family member, a change in a life rhythm, and the like, and further individually optimizes a service proposal and a countermeasure from the next time onward. For example, it contributes to an improvement in a quality of life and a reduction in a feeling of loneliness, such as “propose a walk and exercise because an outing frequency has decreased recently”, “provide nutrition guidance because a meal balance is biased”, and “propose a hobby and an exchange event”. Specific examples of a prompt sentence to be read by the generative AI include “Optimize a proposal and a countermeasure from the next time onward based on a service usage history, health data, and feedback from a family member”, and the like. Specifically, the server includes a continuous learning module implementing a reinforcement learning algorithm. The continuous learning module collects a reaction of a user to a proposal of the generative AI as a reward signal. The continuous learning module gives a positive reward when the user accepts the proposal, and gives a negative reward when the user rejects or ignores the proposal. Input data to the continuous learning module is, for example, tuple data including a vector representation of past proposal content, a context vector indicating a current state of the user, and an acquired reward value. A specific example of input data to the continuous learning module is numerical and categorical data in which proposal content is “recommendation of a walk”, a context is “no outing for three days”, and a reward value is plus one. The continuous learning module inputs the input data to a deep Q-network. The deep Q-network calculates an expected return for a pair of a state and an action. The deep Q-network generates a probability distribution of an optimal action to be selected in a next state as an output. A specific example of output data of the deep Q-network is a numerical vector in which a probability of a walk proposal is 0.7, a probability of a nutrition guidance proposal is 0.2, and a probability of a hobby event proposal is 0.1. The server receives the output data. The server selects an action having the highest probability, and sets it as a parameter of a next service proposal module. The server randomly samples a mini-batch from a periodically accumulated experience replay buffer. The server updates a weight parameter of the deep Q-network by a gradient descent method using a temporal difference error. By this process, unlike a static rule-based system, the system dynamically adapts to a preference and a health condition of a user that change over time. The system thereby has a technical effect of continuously improving an acceptance rate of a proposal and effectively promoting a behavior change of a user. Specific application fields of the system are a personalized healthcare coaching system and an adaptive life support robot. The continuous learning module adopts an elastic weight consolidation method of learning a new pattern while retaining past important experience data in order to prevent catastrophic forgetting. The system adopts a non-conventional processing method of formulating life support as a mathematical optimization problem of reward maximization in a high-dimensional space without depending on a human rule of thumb. The system thereby automatically generates custom-made support perfectly matching characteristics of an individual.

### Specific Use Case

For example, when an elderly person utters “I feel sluggish today and cannot go shopping, I want to order milk and bread” to a communication robot installed in a living space, the communication unit recognizes this utterance with high accuracy by voice and transmits data to the generative AI on the server side. The generative AI simultaneously extracts a request for shopping support and a sign of poor physical condition from the utterance content using a prompt “analyze the utterance content of the elderly person and determine a request, a physical condition change, and an urgency”. The service linkage unit calls an API of an EC site, acquires a product list of milk and bread, selects an optimal product by also referring to a past purchase history and an inventory status, and automatically executes an ordering process. Furthermore, since there was an utterance of poor physical condition, the generative AI detects a change in the health condition using a prompt “extract a service request and a health condition from the utterance content”, and notifies the health condition detection unit. The health condition detection unit transmits data from a heart rate sensor, a body temperature sensor, an acceleration sensor, and the like on the terminal side to the server, and the generative AI performs analysis with a prompt “detect a health abnormality and an activity amount decrease from sensor data, and notify a family member and an administrative agency as necessary”, continues normal life support if there is no abnormality, but automatically notifies a family member and a medical institution if an abnormality is detected, and responds by calling an API of a remote medical examination and an emergency transportation service as necessary. Specifically, the communication unit applies a beamforming process and a noise canceling process to a voice signal acquired using a microphone array. The communication unit transmits clear voice waveform data to the server. The server converts the voice waveform data into text data using a voice recognition model. The server inputs the text data into a large language model subjected to multi-task learning. Input data to the large language model includes, for example, a token array of a text “I feel sluggish today and cannot go shopping, I want to order milk and bread” and a token array of a system prompt. The number of dimensions of the input data to the large language model is, for example, a 4096-dimensional embedding vector. The large language model analyzes a context using a self-attention mechanism. The large language model simultaneously generates a plurality of pieces of structured data for each task as an output. As a specific example of output data of the large language model, a first task output is data in which an intent label is “shopping request” and entities are “milk, bread”, and a second task output is data in which a health condition label is “malaise” and an urgency score is 0.4. The server receives the output data. The server activates the service linkage unit based on the first task output. The service linkage unit transmits a product search query to the API of the EC site, specifies an optimal product ID by calculating a cosine similarity with a past purchase history vector, and transmits an HTTP POST request for order confirmation. At the same time, the server activates the health condition detection unit based on the second task output. The health condition detection unit acquires time-series data for the latest one hour from the heart rate sensor and the body temperature sensor on the terminal side. The health condition detection unit inputs the time-series data into an autoencoder model for abnormality detection. The autoencoder model calculates a reconstruction error of the input data and generates an abnormality score as an output. A specific example of output data of the autoencoder model is numerical data having an abnormality score of 0.75. The server determines whether or not the abnormality score exceeds 0.7 which is a threshold value. When the abnormality score exceeds the threshold value, the server transfers an emergency flag to a subsequent notification module. The notification module transmits a push notification to a terminal of a family member, and calls an API of a remote medical examination system to secure a reservation slot. By this process, the system executes tasks of a plurality of different domains in parallel and asynchronously from one complex utterance. Thereby, the system exerts a technical effect of providing comprehensive support without delay while minimizing an input burden on a user. Specific application fields of the system are a smart home assistant and a medical and nursing care linkage system that require advanced natural language understanding. Unlike sequential information processing by a human, the system adopts an unconventional processing method that integrates multimodal data and multi-task inference. Thereby, the system instantly completes a complex situation judgment.

Also, for example, when an elderly person utters “Please clean”, the communication unit acquires the utterance, and the generative AI extracts a request for a cleaning service with a prompt “analyze the utterance content of the elderly person and determine a request, a physical condition change, and an urgency”. The service linkage unit calls an IoT control API of a robot vacuum cleaner to automatically operate it, and makes a reservation for a cleaning service by a person as necessary. An implementation status of the cleaning service is recorded in the remote monitoring unit, and a family member can confirm “when a parent used the cleaning service”, “an operation status of the robot vacuum cleaner”, and the like in real time with a dedicated application. Specifically, the communication unit specifies a start and an end of an utterance using a voice activity detection algorithm, and cuts out voice data. The server converts the voice data into text, and inputs it into a lightweight transformer model specialized for intent classification. Input data to the transformer model includes, for example, a token array of a short text “Please clean” and context information. The number of dimensions of the input data to the transformer model is, for example, a 512-dimensional vector. The transformer model generates a category ID of an action to be executed and a reliability score as an output. A specific example of output data of the transformer model is numerical data in which a category ID is “IOT_CONTROL_VACUUM_CLEANER” and a reliability score is 0.98. The server receives the output data. When the reliability score exceeds a threshold value, the server transmits an instruction to generate a control command to the service linkage unit. The service linkage unit generates an abstracted control command independent of a model of the robot vacuum cleaner. The service linkage unit converts the control command into a specific IoT protocol, and transmits it to an API endpoint of the robot vacuum cleaner. A specific example of input data to the service linkage unit is key-value data in which a target device ID is “ROBOT_VACUUM_01”, an action is “START_CLEANING”, and a target area is “living room”. The robot vacuum cleaner receives the command, and starts cleaning using an autonomous navigation algorithm. The robot vacuum cleaner transmits status data including position coordinates during operation, a remaining battery level, and a cleaning completed area to the server at a one-second cycle. The remote monitoring unit receives the status data, and stores it in a time-series database. The remote monitoring unit generates drawing polygon data for mapping the status data on a two-dimensional floor map in response to a request from a terminal of a family member. A specific example of output data of the remote monitoring unit is a data structure conforming to a JSON format including a coordinate array indicating a cleaned area and XY coordinates of a current position. By this process, the system converts an ambiguous instruction in a natural language into a specific hardware control command with high accuracy. Thereby, the system exerts a technical effect of eliminating the need for a user to learn a complex device operation and automating maintenance of a living environment. Specific application fields of the system are an integrated control platform for smart home appliances and an autonomous facility management system. The system adopts an unconventional processing method of constructing a closed system from intent understanding to a feedback loop of a device, rather than a mere alternative to a remote controller. Thereby, the system visualizes an operation status of a device and facilitates monitoring from a remote location.

Furthermore, for example, when an elderly person utters “Tell me the news”, the communication unit acquires the utterance, and the generative AI extracts a request for an entertainment service with a prompt “analyze the utterance content of the elderly person and determine a request, a physical condition change, and an urgency”. The service linkage unit calls an API of a news distribution service, acquires the latest news, and provides it to the elderly person by voice or a display on the terminal side. Similarly, when the elderly person utters “Tell me the baseball results”, it calls an API of sports news and provides the latest game results and player information. Specifically, the server implements a retrieval-augmented generation architecture specialized for information retrieval and summary generation. The server receives text-converted utterance data from the communication unit. The server inputs the utterance data into a query encoder. Input data to the query encoder is, for example, a token array of a text “Tell me the baseball results”. The number of dimensions of the input data to the query encoder is, for example, a 768-dimensional dense vector. The query encoder generates a search query vector capturing a semantic feature as an output. The server executes an approximate nearest neighbor search on a vector database of an external news distribution service using the search query vector. The server receives text data and metadata of top five news articles having a high degree of relevance as an output. A specific example of output data of the server is structured data in which an article title is “Yesterday's professional baseball game results”, a body text, and a relevance score is 0.95. The server inputs the acquired text data of the news articles into a large language model. The large language model summarizes it into a plain expression easy for an elderly person to understand and an appropriate length. The large language model generates summary text data as an output. A specific example of output data of the large language model is character string data “In yesterday's game, the local team won five to three. The ace pitcher pitched a complete game.” The server inputs the summary text data into a voice synthesis engine. The voice synthesis engine predicts prosody information from the text, and generates natural utterance waveform data. The server transmits the utterance waveform data to the terminal, and causes a speaker to play it back. At the same time, the server transmits a rendering command for displaying image data of the article on a display of the terminal. By this process, the system instantly extracts information matching a user's intent from a vast amount of web information, converts it into a format with a low cognitive load, and provides it. Thereby, the system exerts a technical effect of eliminating an information gap and promoting social participation of a user. Specific application fields of the system are an interactive infotainment system and an information provision support system for a visually impaired person. The system adopts an unconventional processing method combining a search based on a semantic similarity in a high-dimensional vector space and dynamic content generation by generative AI, rather than a simple search by a keyword. Thereby, the system provides accurate information even for an ambiguous request of a user.

Also, for example, when an elderly person utters “I want to go to a hospital today”, the generative AI extracts a request for medical support with a prompt “analyze the utterance content of the elderly person and determine a request, a physical condition change, and an urgency”, and the service linkage unit sets up a video call with a doctor using an API of a remote medical examination system, and arranges a pick-up and drop-off service of an autonomous vehicle as necessary. A usage history and medical examination content of the medical support are recorded in the data management unit, and a family member can confirm “when a parent used a medical service”, “a main point of medical examination content”, and the like through the remote monitoring unit. Specifically, the server includes a schedule optimization algorithm and a resource allocation engine. The server receives an intent label indicating a request for medical support from the generative AI. The service linkage unit transmits an available slot inquiry request to the API of the remote medical examination system. Input data to the service linkage unit is, for example, a parameter vector including an ID of a target clinical department, a range of a desired date and time, and a priority score of a user. A specific example of input data to the service linkage unit is data in which a clinical department is “internal medicine”, a desired date and time is “this morning”, and a priority score is “high”. The service linkage unit receives matrix data of reservable time slots as an output from the remote medical examination system. The service linkage unit collates the matrix data with existing schedule data of the user, and determines an optimal reservation date and time. The service linkage unit transmits an API request for reservation confirmation, and acquires a session URL for a video call. Furthermore, the service linkage unit transmits a pick-up and drop-off request to a dispatch API of an autonomous vehicle. Input data to the dispatch API is, for example, spatial data including GPS coordinates of a departure place, GPS coordinates of a destination, and a desired arrival time. A specific example of output data of the dispatch API is data in which a dispatch confirmation flag is true, a vehicle ID is “CAR_789”, and a scheduled pick-up time is “10:15 AM”. The server records these pieces of reservation information as a transaction in a secure relational database of the data management unit. After the medical examination is completed, the server inputs text data of an electronic medical record input by a doctor into a large language model. The large language model converts a technical term into plain words, and extracts a main point of the medical examination content. The large language model generates summary text data as an output. The remote monitoring unit transmits the summary text data to a terminal of a family member. By this process, the system cross-sectionally links systems of a plurality of independent service providers, and completely automates a complex arrangement process. Thereby, the system exerts a technical effect of dramatically reducing a physical and psychological burden associated with a user's hospital visit. A specific application field of the system is a next-generation healthcare platform linked with Mobility as a Service. The system adopts an unconventional processing method of formulating an adjustment task performed by a human coordinator as a constraint satisfaction problem and instantly solving it using a mathematical optimization solver. Thereby, the system avoids resource conflict and guarantees reliable service provision.

Furthermore, for example, when a fall detection sensor on the terminal side detects a rapid acceleration change, a sensor linkage unit transmits data to the server, and the generative AI determines an urgency with a prompt “detect a health abnormality, a fall, and an activity amount decrease from sensor data, and notify a family member and an administrative agency as necessary”, automatically notifies a family member and an administrative agency when an abnormality is recognized, and calls an API of an emergency transportation service to arrange an ambulance. Specifically, the fall detection sensor on the terminal side incorporates a three-axis acceleration sensor and a gyro sensor. The terminal executes a lightweight machine learning model in an edge computing environment. The terminal cuts out raw data acquired from the sensor at a sampling rate of 100 Hz by a sliding window method. The terminal extracts feature amounts such as variance, kurtosis, and a frequency component from data in the window. The terminal inputs the extracted feature amount vector into a support vector machine. The terminal calculates an initial score indicating a possibility of a fall as an output. The terminal transmits the raw data of the window to the server only when the initial score exceeds a certain threshold value. The server inputs the received raw data into a deep convolutional neural network. Input data to the deep convolutional neural network is, for example, a two-dimensional tensor composed of three-channel time-series waveform data. The number of dimensions of the input data to the deep convolutional neural network is, for example, a 3×300 matrix. The deep convolutional neural network hierarchically extracts spatial and temporal features, and generates a final fall probability and an urgency class as an output. A specific example of output data of the deep convolutional neural network is numerical and categorical data in which a fall probability is 0.99 and an urgency class is “severe of level 5”. The server receives the output data. When determining that the urgency class is the highest level, the server immediately executes a subsequent emergency response protocol. The server transmits an override notification accompanied by a vibration and a warning sound to a terminal of a family member. At the same time, the server packages and transmits GPS coordinates of a user, medical history data, and sensor waveform data at the time of a fall to an API of an emergency transportation service. By this process, the system reduces a false alarm to the utmost limit while saving a communication band by combining primary screening on an edge side and high-precision secondary determination on a cloud side. Thereby, the system exerts a technical effect of activating a lifesaving measure with a response speed in milliseconds for a true emergency. Specific application fields of the system are an advanced monitoring system using a wearable device and a safety management infrastructure of a smart home. The system adopts an unconventional processing method based on pattern recognition of a waveform, rather than a simple threshold determination of acceleration. Thereby, the system identifies a daily action and an actual fall with high accuracy.

Also, for example, when a family member makes an inquiry such as “How much did food expenses cost this month?” or “I want to confirm a recent health condition in detail” from a dedicated application, the generative AI extracts necessary information from the data management unit with a prompt “extract appropriate information in response to a remote instruction or an inquiry from a family member, and answer in an easy-to-understand manner”, and presents it to the family member in an easy-to-understand manner using a graph or a list. Specifically, the server implements a Text-to-SQL technology which is a database query generation function by a natural language. The server receives an inquiry text in a natural language transmitted from a terminal of a family member. The server inputs the inquiry text into a large language model. Input data to the large language model is, for example, a prompt including a token array of a text “How much did food expenses cost this month?” and database schema information of the data management unit. The number of dimensions of the input data to the large language model is, for example, a 2048-dimensional embedding vector. The large language model understands a structure of a database, and generates an executable SQL query character string as an output. A specific example of output data of the large language model is character string data “SELECT SUM(amount) FROM settlement_history WHERE category=‘food expenses’ AND month=‘current month’”. The server issues the SQL query to a relational database engine of the data management unit. The server receives numerical data of an aggregation result as an output from the database. The server inputs the numerical data and time-series data regarding “recent health condition” into the large language model again. The large language model converts these pieces of raw data into an explanatory text in a natural language easy for a human to read. The large language model generates an explanatory text and structured data for drawing a graph as an output. A specific example of output data of the large language model is a text in which an explanatory text is “Food expenses for this month are 35,000 yen. The health condition is generally good, but sleep time has been on a decreasing trend since last week.”, and data for drawing a graph is a one-dimensional array indicating a transition of sleep time. The server transmits the output data to a terminal of a family member. The terminal of the family member renders a text and a dynamic line graph on a user interface of a dedicated application. By this process, the system enables even a user who does not have knowledge of a specialized database operation to execute complex data analysis in an intuitive interactive format. Thereby, the system exerts a technical effect of dramatically improving data accessibility and reducing a nursing care burden on a family member. Specific application fields of the system are an interactive data visualization tool and a personal assistant application. The system adopts an unconventional processing method of generating data extraction and visualization on the fly in accordance with a dynamic intent of a user, rather than providing a fixed dashboard. Thereby, the system flexibly responds to an infinite variation of inquiries.

Specific examples of a prompt sentence to be read by generative AI necessary for implementing the present invention include “analyze the utterance content of the elderly person and determine a request, a physical condition change, and an urgency”, “extract a service request and a health condition from the utterance content”, “propose an optimal life support service based on a past service usage history and health data”, “detect a health abnormality, a fall, and an activity amount decrease from sensor data, and notify a family member and an administrative agency as necessary”, “extract appropriate information in response to a remote instruction or an inquiry from a family member, and answer in an easy-to-understand manner”, and “optimize a proposal and a countermeasure from next time onward based on a service usage history, health data, and feedback from a family member”. Specifically, the server includes a dynamic prompt generation module that automates prompt engineering. The dynamic prompt generation module embeds a dynamic variable into a base prompt template in accordance with a state of the system, a context of a user, and a type of a task to be executed. Input data to the dynamic prompt generation module is, for example, a context dictionary including a task identifier, a latest action history vector of a user, and real-time environmental data from a sensor. A specific example of input data to the dynamic prompt generation module is key-value data in which a task identifier is “intent extraction”, an action history is “no activity for the latest one hour”, and environmental data is “room temperature 10 degrees”. The dynamic prompt generation module analyzes these pieces of input data, and synthesizes an optimal instruction sentence, a constraint condition, and an output format designation for maximizing inference accuracy of a large language model. The dynamic prompt generation module generates an optimized prompt character string as an output. A specific example of output data of the dynamic prompt generation module is character string data “You are an excellent nursing care assistant. Please analyze the following utterance content and determine a request, a physical condition change, and an urgency. Please be sure to output in a structured data format having a designated key.” The server combines the prompt character string and input data of a user, and inputs them into a large language model. The large language model strictly follows an instruction of the prompt, and outputs an inference result in a format parsable by a subsequent system. The server includes a parser module that verifies whether or not a format of the output inference result is correct. When detecting a format error, the parser module automatically generates a correction prompt including error content, and requests a large language model to perform re-inference. By this process, the system suppresses hallucination and fluctuation of an output format of a large language model, and enables deterministic system linkage. Thereby, the system exerts a technical effect of safely incorporating an output of AI into a mission-critical business process. Specific application fields of the system are an autonomous AI agent system for an enterprise and a robot control infrastructure. The system adopts an unconventional processing method of implementing dynamic generation of a prompt in accordance with a context and a self-healing loop, rather than a human manually adjusting a prompt. Thereby, the system realizes high adaptability to an environmental change and robustness.

(Application Example 1) The flow of the specific processing in Application Example 1 will be described. Each part of the system described below is realized by the data processing device 12 and the smart device 14. Additionally, the data processing device 12 is referred to as a “server,” and the smart device 14 is referred to as a “terminal.”

### Embodiment for Carrying out the Present Invention

An embodiment of the present invention is a comprehensive city life support system that integrally and autonomously realizes life support, health management, public service optimization, disaster response, and efficient operation of city infrastructure for each individual resident of an entire city on a city scale. The system has a configuration in which a communication terminal and various IoT sensors installed in every place constituting a city, such as each home, apartment complex, public facility, transportation, medical institution, commercial facility, administrative counter, educational institution, entertainment facility, outdoor plaza, underground mall, parking lot, station, bus stop, taxi stand, sidewalk, intersection, traffic light, street light, garbage collection point, evacuation shelter, and disaster response base in a city, are networked and linked with a server in a city center. Specifically, the system adopts a large-scale distributed processing architecture in which edge computing, fog computing, and cloud computing are hierarchically integrated. The system defines a communication terminal and an IoT sensor group innumerably arranged in a city space as an edge node of a first layer. The system defines a gateway device installed for each region or facility as a fog node of a second layer. The system defines a high-performance parallel computing cluster arranged in a data center in a city center as a cloud server of a third layer. Input data to the cloud server is, for example, a multidimensional data stream transmitted every second from millions of sensors in an entire city. A specific example of input data to the cloud server is a huge amount of log data including a numerical vector indicating a traffic volume of each intersection, a time-series array indicating power consumption of each public facility, and a service request from each home. The cloud server ingests and aggregates these pieces of input data in real time using a distributed stream processing engine. The cloud server inputs the aggregated data into a digital twin model of an entire city. The digital twin model models a physical connection relationship of city infrastructure and dynamics of a resident using a graph neural network. The digital twin model generates an optimal resource allocation matrix of an entire city and an infrastructure control command as an output. A specific example of output data of the digital twin model is a control flag indicating a green time extension of a traffic light in a specific area, and a parameter vector indicating load balancing of a power grid. The cloud server transmits the output data to a corresponding edge device via each fog node. By this process, the system autonomously executes overall optimization of an entire city, which is impossible with an individual siloed system. Thereby, the system exerts an extremely large-scale technical effect of maximizing energy efficiency, eliminating traffic congestion, and rapidly relocating resources during a disaster. Specific application fields of the system are a city operating system of a next-generation smart city and a wide-area disaster response platform. The system adopts an unconventional processing method of hierarchical distributed processing based on spatial and temporal locality, rather than centralized batch processing. Thereby, the system prevents depletion of a network band, and realizes ultra-low latency control on a city scale.

The communication terminal takes various forms such as a voice recognition robot, a smart speaker, a tablet, digital signage, a wearable device, a smartphone, an in-vehicle terminal, a public guidance terminal, an electronic bulletin board, and an emergency call button. For example, in a home, a terminal is installed in a living room, a bedroom, a bathroom, a kitchen, an entrance, and the like, and acquires an utterance, an action, a facial expression, a gesture, a touch operation, and vital data of a resident. For example, in a common area of an apartment complex, a terminal is installed in an elevator hall, a corridor, a garbage storage area, a meeting room, a bicycle parking lot, and the like, and receives a request, a complaint, an emergency call, a facility usage application, and the like of a resident. For example, in a public facility, a terminal is installed in a library, a gymnasium, a cultural hall, a government office, a hospital, a welfare center, a children's center, a nursing home, a nursery school, a school, and the like, and performs guidance, reservation, consultation, health check, evacuation guidance during an emergency, and the like for a user. For example, in transportation, a terminal is installed in a station, a bus stop, a taxi stand, and a vehicle, and performs time guidance, transfer guidance, congestion status notification, fare payment, lost property inquiry, barrier-free support, operation information provision during a disaster, and the like. For example, a terminal is also installed in a commercial facility, a restaurant, a shopping mall, a supermarket, a convenience store, a drugstore, a dry cleaner, a delivery locker, an ATM, a coin laundry, and the like, and performs product guidance, ordering, settlement, point management, coupon distribution, congestion status notification, lost property notification, emergency evacuation guidance, and the like. Specifically, the communication terminal is equipped with a multimodal interface and an edge AI processor. The communication terminal simultaneously acquires raw data of a plurality of modalities from a built-in microphone, camera, touch panel, and various vital sensors. The communication terminal locally executes preprocessing and feature extraction on the raw data using an edge AI processor. Input data to the edge AI processor is, for example, a continuous tensor of a video frame of full HD resolution, and voice waveform data sampled at 48 kHz. The number of dimensions of the input data to the edge AI processor is, for example, an image tensor of 3 channels×1080×1920. The edge AI processor extracts skeleton coordinates of a person and a feature amount of a facial expression from a video using a lightweight convolutional neural network. The edge AI processor generates a compressed feature amount vector and recognized text data as an output. A specific example of output data of the edge AI processor is a floating-point array indicating skeleton coordinates, categorical data indicating that a facial expression is “confusion”, and a text character string of utterance content. The communication terminal discards raw data from a viewpoint of privacy protection, encrypts only the feature amount vector, and transmits it to a cloud server. The cloud server integrally analyzes the received feature amount vector, and deeply understands an intent and a situation of a user. The cloud server outputs a personalized response and guidance through a display and a speaker of the terminal based on an analysis result. By this process, the system dramatically reduces a data transfer amount to a cloud side, and prevents network congestion. Thereby, the system exerts a technical effect of maintaining real-time interaction even in an environment where millions of terminals operate simultaneously. Specific application fields of the system are an unmanned concierge system in a public space and an information provision terminal based on universal design. The system adopts an unconventional processing method of temporally synchronizing and fusing multimodal data such as vision, hearing, and touch, without depending on a single input source. Thereby, the system achieves extremely high recognition accuracy even in a noisy public environment.

The IoT sensor covers a wide range, such as a fall detection sensor, a heart rate sensor, a blood pressure sensor, a body temperature sensor, a respiration sensor, a sleep sensor, an activity amount sensor, a door opening/closing sensor, a window opening/closing sensor, an illuminance sensor, a temperature sensor, a humidity sensor, an air quality sensor, a carbon dioxide sensor, a noise sensor, a vibration sensor, a water leakage sensor, a smoke sensor, a fire sensor, a gas leak sensor, a congestion degree sensor, a people counting sensor, a face recognition camera, a license plate recognition camera, a traffic volume sensor, a traffic light control sensor, a street light control sensor, a garbage full sensor, and a disaster detection sensor (earthquake, tsunami, typhoon, heavy rain, flood, landslide, volcanic eruption, lightning strike, power outage, water outage, communication failure, etc.). These sensors constantly monitor a health condition and an action of a resident, an operation status of city infrastructure, an environmental change, a disaster sign, a crime risk, and the like, and transmit data to a server in real time. Specifically, the IoT sensor group communicates with a gateway or directly with a cloud server using a low power wide area network technology. The IoT sensor group converts an analog physical quantity acquired by each into digital data by a built-in AD converter. The IoT sensor group applies calibration and filtering processing of an abnormal value to the digital data. Output data from the IoT sensor group is, for example, payload data including an identifier unique to a sensor, a time stamp, and a measurement value. A specific example of output data from the IoT sensor group is structured data in which a sensor ID is “ENV_SENSOR_001”, a time stamp is an epoch second, a carbon dioxide concentration is 1200 ppm, and a temperature is 25 degrees. The cloud server continuously writes these huge amounts of payload data into a time-series database. The cloud server inputs the time-series data into an autoencoder-based neural network specialized for abnormality detection. Input data to the neural network is, for example, a multivariate time-series tensor acquired in the past one hour from hundreds of sensors installed in a specific area in a city. The number of dimensions of the input data to the neural network is, for example, matrix data of 100×3600. The neural network has learned a distribution of data in a normal state, and calculates a reconstruction error of input data. The neural network generates a vector of an abnormality score for each sensor stream as an output. A specific example of output data of the neural network is numerical data in which an abnormality score of a specific water level sensor is 0.95 and an abnormality score of a vibration sensor is 0.88. The cloud server receives the output data. When the abnormality score exceeds a threshold value, the cloud server transfers an alert signal to a subsequent event correlation analysis module. By this process, the system integrates data from innumerable sensors spread throughout an entire city, and issues an early warning before a local abnormality spreads to the entire city. Thereby, the system exerts a technical effect of minimizing downtime of city infrastructure and proactively ensuring safety of a resident. Specific application fields of the system are an integrated monitoring center of a smart city and a wide-area infrastructure predictive maintenance system. The system adopts an unconventional processing method of modeling a complex correlation among a wide variety of sensors in a high-dimensional space, without depending on a threshold determination of an individual sensor. Thereby, the system specifies a complex abnormal event undetectable by a single sensor with high accuracy.

On the server side, generative AI constantly operates and analyzes a request, a health condition, a life pattern, an emergency, a change in a city environment, an operation status of city infrastructure, a disaster risk, a crime risk, a service demand of the entire city, an abnormal tendency, and the like of each individual resident in a multifaceted manner and in chronological order. For example, when a resident utters “I am not feeling well today, so I cannot take out the garbage”, the AI simultaneously extracts the poor physical condition and the garbage collection request, arranges an individual collection in cooperation with a waste management system, automatically notifies a health management center and a family member, and arranges a remote medical examination and a nurse dispatch as necessary. For example, when a resident utters “Tell me the timetable of the nearest bus stop”, it automatically guides a real-time bus timetable, a congestion degree, a ride reservation, and an arrangement of a barrier-free vehicle in cooperation with a city transportation system. For example, when a resident utters “I want to go shopping”, it arranges everything from a product proposal to ordering and delivery collectively in cooperation with a nearby commercial facility and an e-commerce site. For example, when a resident utters “I want to perform an administrative procedure”, it automatically executes everything from a reservation of an administrative counter and guidance on necessary documents to support for an online application. For example, when a resident utters “I want to hold an event in the park”, it collectively supports an event application, a venue reservation, participant recruitment, public relations, guidance on the day, security, cleaning, and cleanup. Specifically, the server includes an orchestration platform integrating a complex event processing engine and a large language model. The server continuously monitors an event stream arriving asynchronously from terminals and sensors throughout the city. When receiving a request in a natural language from a resident, the server inputs the text data to the large language model. Input data to the large language model includes, for example, a token array of a text “I am not feeling well today, so I cannot take out the garbage” and a profile vector of the resident. The number of dimensions of the input data to the large language model is, for example, a 4096-dimensional embedding vector. The large language model deeply understands a context and generates a data structure of a directed acyclic graph defining a plurality of tasks to be executed as an output. As a specific example of output data of the large language model, node A is “individual collection API call to the waste management system”, node B is “notification API call to the health management center”, and node C is “remote medical examination reservation API call”, and it is data in which a dependency relationship between nodes is defined. The server receives the directed acyclic graph. The server executes each task in parallel or in series in accordance with a topological sort of the graph. The server transmits a payload including an address coordinate of the resident and a collection request flag to an API of the waste management system. The server transmits a query including a current location coordinate and a destination coordinate to an API of the city transportation system, and receives a response including an optimal route and an estimated arrival time of a vehicle as an output. The server integrates these execution results and generates a response text to the resident using a natural language generation model. The server transmits the response text to the terminal and provides feedback by voice or screen display. By this processing, the system cross-sectionally links a plurality of administrative services and private services for a single request, and provides a one-stop problem solution. The system thereby breaks down the walls of vertically segmented administration, and exerts a technical effect of dramatically improving the convenience of residents. Specific application fields of the system are a city OS and an integrated resident service portal in a Super City initiative. The system adopts a non-conventional processing method of dynamically generating and executing a workflow in accordance with an intention of a resident and a real-time situation of a city, rather than a predefined static workflow. The system thereby provides a flexible and optimal service for a request of a resident having infinite variations.

As a specific example based on data from an IoT sensor, when a fall detection sensor detects a fall in a corridor of an apartment complex, the AI immediately calls an API of an emergency transportation service and automatically notifies a family member, a medical institution, a manager, and a neighboring resident. When an air quality sensor falls below a reference value, the AI automatically notifies a corresponding resident and a facility manager, and automatically controls a ventilation system and an air conditioning facility. When a noise sensor detects an abnormal value, the AI performs a cause analysis, notifies a resident and a manager as necessary, and performs identification of a noise source and an improvement proposal. When a congestion degree sensor detects congestion in a public transportation or a commercial facility, the AI notifies a user and an operator in real time, and automatically proposes and executes a congestion mitigation measure (for example, an admission restriction, a staggered use, an extra flight operation, guidance, coupon distribution, and the like). When a disaster detection sensor detects an earthquake, heavy rain, a flood, a fire, a power outage, and the like, the AI automatically executes an evacuation instruction, an evacuation shelter guidance, provision of a priority service during a disaster, and infrastructure control (for example, emergency control of a traffic light, lighting of a street light, arrangement of a water truck and a power generation vehicle, and the like). Specifically, the system includes an edge and cloud cooperative inference engine that processes multidimensional time-series data streamed from a plurality of IoT sensors in real time. The inference engine does not depend on human visual monitoring or a simple rule-based threshold determination. The inference engine executes feature extraction in a high-dimensional space. Thereby, the system realizes highly accurate event detection even in a complex environment. The system achieves both saving of a communication band and advanced integrated analysis in a cloud. In a fall detection process, the inference engine receives time-series tensor data from a 3-axis acceleration sensor and a depth camera as an input. This input data includes an image tensor with a frame rate of 30 fps and a 128-dimensional skeleton coordinate vector. The inference engine analyzes these pieces of input data using a spatiotemporal convolutional neural network and a long short-term memory network. The inference engine outputs a fall probability score and a posture classification label. For example, the inference engine outputs a fall probability score of 0.95. The system executes a threshold determination on this output result. The system automatically executes an API call of an emergency transportation service when the score exceeds a specified value. In an air quality management process, the inference engine receives a multivariate time-series array such as a CO2 concentration, PM2.5, and a volatile organic compound as an input. The inference engine executes an analysis using a large language model-based time-series prediction architecture equipped with a self-attention mechanism. The inference engine outputs a future air quality deterioration probability distribution. For example, the inference engine outputs a predicted value that a probability that the CO2 concentration exceeds a reference value after 30 minutes is 85 percent. The system transmits this output result to a subsequent control module. The control module generates a control signal for adjusting an air volume and a ventilation mode of an air conditioning facility. In a noise detection process, the inference engine receives a two-dimensional spectrogram tensor of Mel-frequency cepstral coefficients converted from voice waveform data as an input. The inference engine extracts a feature map using a multi-layer convolutional neural network. The inference engine outputs a classification label of a noise source and a reliability score. For example, the inference engine outputs a classification label such as a construction sound, a human scream, and a vehicle passing sound. The system executes a noise source identification process using this output result. In a congestion degree detection process, the inference engine receives a high-resolution image tensor from a surveillance camera and a MAC address hash list obtained from a probe request of wireless communication as an input. The inference engine executes an analysis using an object detection algorithm and a density estimation network. The inference engine outputs an estimated number of people vector for each area and a congestion degree heat map. For example, the inference engine outputs numerical data that the number of staying people in a specific area is 150. The system inputs this output result to a dynamic routing algorithm. The system triggers a detour route display on a digital signage and a coupon distribution API for congestion mitigation. In a disaster detection process, the inference engine receives waveform data of a seismometer, a continuous numerical vector of a water level sensor, and a binary signal of a smoke detector as an input. The inference engine executes an analysis using an anomaly detection autoencoder using multimodal learning. The inference engine outputs polygon data indicating a severity level and an influence range of a disaster. For example, the inference engine outputs a numerical value indicating a warning level of level 5 and a coordinate group of an inundation prediction area. The system generates an infrastructure control signal based on this output result. The system immediately executes physical hardware control such as fixing a traffic light to red and lighting a street light at maximum brightness. By these processes, the system exerts a remarkable technical effect of autonomous optimization of city infrastructure and improvement of safety of residents.

The data management unit centrally manages a transition of a health condition for each resident, a service usage history, an emergency response history, an occurrence history of an environmental abnormality, an operation status of city infrastructure of the entire city, a disaster occurrence history, a crime occurrence history, a traffic accident history, an event holding history, an administrative procedure history, an energy consumption history, a garbage discharge history, a recycling history, a resident satisfaction survey result, and the like, and provides appropriate information to a family member, an administrative agency, a medical institution, an infrastructure operator, police, a fire department, an educational institution, a research institution, a private business operator, and the like as necessary. The data management unit has functions such as data encryption, access authority management, backup, data erasure, provision of an interface for data analysis, privacy protection, security monitoring, cyber attack countermeasure, legal compliance, anonymization processing, and data distribution management. Specifically, the data management unit adopts a secure data lake architecture based on a distributed database and blockchain technology. The data management unit prevents siloing of enormous city data. The data management unit enables a high-speed cross-domain search. The data management unit normalizes various input history data as a multidimensional vector including a time stamp, a geotag, and a user identifier. The data management unit inputs this normalized multidimensional vector group to a graph neural network and an autoencoder. The data management unit outputs a feature vector, an anomaly score, and a weight matrix of a correlation in a latent space. For example, the data management unit outputs a weight matrix indicating a hidden correlation between a specific weather condition and a traffic accident occurrence rate. The system executes an optimization simulation of city planning using this output result. In privacy protection and anonymization processing, the data management unit executes a synthetic data generation algorithm using a generative adversarial network. The data management unit receives a dataset including original personal information as an input. A generator network of the data management unit generates fake data from a random number vector. A discriminator network of the data management unit discriminates between real data and fake data. The data management unit trains these two networks competitively. The data management unit outputs a synthetic dataset in which an individual cannot be identified while maintaining statistical properties. For example, the data management unit outputs health condition transition data and service usage history data of a non-existent resident. The system uses this synthetic dataset as data for provision to a research institution and a private business operator. Thereby, the system completely eliminates a risk of privacy infringement. In encryption processing, the data management unit adopts a homomorphic encryption method. The data management unit inputs a data tensor in an encrypted state to a neural network. The data management unit executes an inference operation without performing a decryption process. The data management unit outputs an encrypted inference result. Thereby, the system physically eliminates a risk of data leakage on a main memory. In access control, the data management unit implements a dynamic access control model based on a zero trust architecture. The data management unit receives a context of an access request source as an input. This context includes a time-series array of an IP address, a security state of a device, and a request time zone. The data management unit calculates an unauthorized access probability using a random forest and a gradient boosting decision tree. The data management unit outputs a binary flag indicating access permission or denial. For example, the data management unit calculates that the unauthorized access probability is 0.85 and outputs a denial flag. The system dynamically updates a packet filtering rule of a network switch and a firewall based on this output result. By these advanced encryptions and AI-driven security monitoring, the system prevents data falsification and information leakage. The system exerts a technical effect of dramatically improving the robustness of the entire system.

The user interface unit provides various interfaces such as voice, video, touch, gesture, text, AR, VR, web, application, chatbot, and API linkage so that various users such as a resident, a family member, an administrative official, a medical worker, an infrastructure operator, a security guard, a volunteer, a researcher, a tourist, an event organizer, and a business operator can intuitively use the system. For example, when a resident inputs a request such as “I want to confirm a health condition of a parent”, “I want to see a garbage collection history of my home”, “I want to know an air quality and a noise situation in the neighborhood”, “I want to acquire evacuation shelter information during a disaster”, and “I want to apply for participation in an event” from an application, the AI extracts necessary information and presents it in an easy-to-understand manner by a graph, a list, a map, a video, voice, an AR display, and the like. An administrative official can confirm a service usage status and an abnormal tendency of the entire city on a dashboard, and utilize them for an additional response, resource allocation, policy making, resident explanation, press release, disaster response headquarters operation, and the like. A medical worker can refer to health data and a life history of a patient, and utilize them for a remote medical examination, health guidance, medication management, rehabilitation support, an emergency response, a home medical care plan, and the like. An infrastructure operator can refer to an operation status and an abnormality detection history of a facility, and utilize them for a maintenance plan, a failure response, energy optimization, a facility renewal plan, disaster recovery, preventive maintenance, and the like. Specifically, the user interface unit includes a multimodal interface engine that integrally processes input data of various modalities. The engine converts voice waveform data into a Mel-frequency cepstral coefficient feature vector. The engine converts video data into a three-dimensional RGB tensor in units of frames. The engine inputs these feature vectors and tensors to a large language model having a self-attention mechanism. The large language model maps the input multimodal data to the same latent space. The large language model outputs an intention classification score indicating a user's intention and a probability distribution of an extracted entity. For example, assume a case where input data is voice data “I want to confirm a health condition of a parent”. The large language model outputs a reliability score of 0.98 for an intention label “health condition inquiry”. The large language model outputs a probability value of 0.95 for a target entity “parent”. The user interface unit issues a query to a back-end database based on this output result. The user interface unit converts the acquired time-series vital data into a JSON object for dynamic graph drawing. The user interface unit transmits this object to a terminal of the user to render it on a screen. By this processing, the system eliminates a conventional process in which a human manually searches a database. The system directly generates a structured query from an ambiguous input of a user. The system significantly reduces latency of information retrieval. The system executes similar processing also in dashboard generation for an administrative official. The system aggregates millions of service usage logs of the entire city in real time. The system holds an aggregation result as multidimensional array data. The system inputs this multidimensional array data to a clustering algorithm. The system automatically extracts a cluster indicating an abnormal usage tendency. The system converts coordinate data of the extracted cluster into a heat map image tensor. The system superimposes and displays this heat map on the dashboard. Thereby, the system enables an administrative official to visually and immediately grasp an abnormal state of the city. For a medical worker, the system generates a time-series tensor integrating past electronic medical record data and current vital data of a patient. The system inputs this time-series tensor to a recurrent neural network. The system calculates a prediction score indicating a future health risk. The system push-notifies an emergency alert to a terminal of the medical worker when the prediction score exceeds a predetermined threshold. For an infrastructure operator, the system acquires an operation log of each facility as a time-series vector. The system inputs this time-series vector to an anomaly detection model. The system outputs an anomaly score indicating a degree of deviation from a normal state. The system automatically generates an optimization schedule of a maintenance plan based on the anomaly score. With these configurations, the system provides intuitive and immediate information access to various users. The system improves operability of the entire system. The system technically supports a decision-making process in each specialized field.

The generative AI comprehensively analyzes an attribute of a resident, a past usage history, a health condition, a life rhythm, an environmental change, a situation of the entire city, an operation status of city infrastructure, a disaster risk, a crime risk, a service demand, resident satisfaction, a policy effect, a social trend, weather information, traffic information, energy supply and demand, population dynamics, a tourism trend, an economic indicator, an educational indicator, a medical indicator, a welfare indicator, an environmental indicator, disaster statistics, crime statistics, and the like, and automatically generates and executes a service proposal and information provision optimized for each individual resident, optimal operation of the entire city, a rapid response during a disaster, infrastructure efficiency improvement, social problem solving, and sustainable city management. For example, when a resident is elderly and a frequency of going out is decreasing, it individually optimizes and proposes a walk, an exercise, an exchange event, a health class, a hobby activity, a volunteer activity, regional community participation, an online exchange, a remote medical examination, a meal delivery service, shopping support, a monitoring service, a family contact, safety confirmation, life rhythm improvement, rehabilitation support, and the like. When a balance of a meal is biased, it automatically proposes nutritional guidance, a meal delivery service, a recipe proposal, shopping list creation, cooking support, a meal record, a health consultation, meal event participation, and the like. When utterances indicating a feeling of loneliness are increasing, it automatically proposes an exchange opportunity with a family member and a regional community, an online exchange, a hobby circle, event participation, a volunteer activity, a pet robot, a mental health consultation, counseling, psychological support, and the like. During a disaster, it notifies a resident of a congestion status and safety information of an evacuation shelter in real time, and collectively supports guidance on an evacuation route and a priority service, safety confirmation, a family contact, disaster information provision, disaster recovery support, volunteer recruitment, relief supply distribution, medical support, infrastructure recovery, an administrative response, a press release, and the like. Specifically, the generative AI is configured as a transformer-based deep learning model having hundreds of billions of parameters. The generative AI integrally processes heterogeneous mixed data collected from the entire city. The system normalizes attribute data, past usage history data, and health condition data of a resident, respectively. The system converts the normalized data into a 512-dimensional dense vector. The system acquires environmental data such as weather information, traffic information, and energy supply and demand data as a time-series numerical array. The system inputs these numerical arrays to a one-dimensional convolutional neural network to extract an environmental feature vector. The system combines the feature vector of the resident and the environmental feature vector. The system inputs the combined vector to an encoder of the generative AI. The generative AI calculates a hidden correlation between respective pieces of data using a self-attention mechanism. The generative AI outputs a text token sequence indicating an optimal service proposal and structured data of an execution command through a decoder. For example, assume a case where resident data of “age 85”, “number of times of going out in the past week is 0”, and “step count data is 500 steps on average per day” is given as input data. The generative AI analyzes this input vector. The generative AI generates an action code “SVC001” indicating “proposal of a meal delivery service” and an action code “SVC002” indicating “guidance on an online exchange event” as an output. The generative AI outputs a recommendation score for each action in a range of 0.0 to 1.0. The system automatically selects a proposal having a recommendation score of 0.8 or more. The system push-notifies the selected proposal to a terminal of the resident. Also, assume a case where nutritional data of “carbohydrate ratio of a meal is 80 percent” and “protein intake is less than a reference value” is given as input data. The generative AI outputs specific recipe data and a purchase list of ingredients for supplementing a lacking nutrient in a JSON format. The system transmits this JSON data to an inventory management system of a nearby supermarket. The system automatically executes a layaway process of the ingredients. Furthermore, assume a case where voice text data of “lonely” and “not talking to anyone” is given as input data. The generative AI calculates a loneliness score using an emotion analysis model. The system automatically issues an API call of a visit request to a regional volunteer management system when the loneliness score exceeds a threshold. During a disaster, the system acquires capacity data and current evacuee number data of each evacuation shelter in real time. The system inputs these pieces of data to a graph neural network. The system calculates a congestion prediction score of each evacuation shelter. The system identifies an evacuation shelter having a low congestion prediction score. The system generates a coordinate list of an optimal evacuation route based on location information data of the resident. The system transmits this coordinate list to a map application on a smartphone of the resident to draw the route. In a learning process of the generative AI, the system uses a past service proposal history and its acceptance result as teacher data. The system calculates a loss using a cross-entropy error function. The system optimizes a weight of a network by an error backpropagation method. With this configuration, the system realizes data-driven decision making that does not depend on human intuition or experience. The system optimizes allocation of city resources. The system eliminates waste of calculation resources and dramatically improves processing efficiency of the entire system.

As described above, the embodiment of the present invention centrally and autonomously realizes life support of a resident, health management, public service optimization, a disaster response, efficient infrastructure operation, social problem solving, and sustainable city management on a city scale, and can dramatically enhance QOL improvement of each individual resident, safety, efficiency, and sustainability of the entire city, happiness improvement of the entire society, economic revitalization, environmental conservation, disaster resilience, social inclusion, and innovation creation. Specifically, the system adopts a hierarchical computing architecture that links edge devices distributed throughout the city and a central cloud server. The system executes a lightweight inference model in each edge device. The system completes primary processing requiring real-time performance on the edge side. The system delegates secondary processing requiring advanced context understanding to the generative AI on the cloud side. By this load balancing mechanism, the system significantly reduces communication traffic to the cloud server. The system prevents depletion of a network band. The system suppresses a response delay of the entire system to a millisecond unit. The system applies a federated learning algorithm when processing highly private data such as vital data and an action log of a resident. The system encrypts and transmits only gradient information of a model calculated in each terminal without transmitting raw data to the cloud. The system aggregates the gradient information on the cloud side and updates a global model. Thereby, the system technically eliminates a risk of leakage of personal information. The system realizes secure data utilization. The system continuously monitors operation data of city infrastructure. The system captures a failure sign of a facility using an anomaly detection model. The system automatically generates a schedule of preventive maintenance before a failure occurs. The system minimizes downtime of infrastructure. The system inputs energy consumption data to a reinforcement learning agent. The system predicts power demand of the entire city. The system dynamically optimizes power supply in cooperation with a smart grid. By integrating these technical processes, the system achieves performance improvement of a computer system itself beyond mere business automation. The system ensures scalability to process an enormous data stream without delay. The system provides high reliability and availability in various use cases as a foundation system of a smart city.

### System Configuration

The system according to the present embodiment includes a communication unit, a city service linkage unit, an environment and health monitoring unit, an emergency response unit, a data management unit, a user interface unit, a sensor linkage unit, and an AI analysis unit. The communication unit acquires an utterance, an action, a facial expression, a gesture, a touch operation, and vital data of a resident in real time using various terminals such as a voice recognition robot, a smart speaker, a tablet, a digital signage, a wearable device, a smartphone, an in-vehicle terminal, a public guidance terminal, an electronic bulletin board, and an emergency call button installed in each home, apartment complex, public facility, transportation, medical institution, commercial facility, administrative counter, educational institution, entertainment facility, outdoor plaza, underground mall, parking lot, station, bus stop, taxi stand, sidewalk, intersection, traffic light, street light, garbage collection point, evacuation shelter, disaster response base, and the like in a city, and, for example, acquires an utterance such as “I am not feeling well today, so I cannot take out the garbage”, “I want to go shopping”, and “Call an ambulance” in a home, receives a request and a complaint such as “The corridor is dark”, “The noise is loud”, and “I want to request cleaning of the garbage area” in a common area of an apartment complex, realizes guidance and a reservation such as “Tell me the timetable of the nearest bus stop”, “I want you to guide me to the event venue”, and “I want to reserve a library book” in a public facility, information provision and support such as “transfer guidance”, “congestion status notification”, and “barrier-free support” in transportation, and a service such as “product guidance”, “ordering”, “settlement”, and “lost item notification” in a commercial facility. Specifically, the system adopts a microservice architecture in which a plurality of independent processing modules communicate via a high-speed internal bus and a secure network protocol. The communication unit controls a voice signal processing circuit and an image processing processor incorporated in each terminal. The communication unit converts an analog voice signal acquired from a microphone array into digital waveform data at a sampling rate of 16 kilohertz. The communication unit applies a noise canceling process and an echo canceling process to this waveform data. The communication unit converts the preprocessed waveform data into an acoustic feature tensor. The communication unit inputs this acoustic feature tensor to a recurrent neural network for voice recognition. The neural network outputs a probability distribution of phonemes from the input tensor. The communication unit generates a most likely text character string using a Viterbi search algorithm. The communication unit extracts a video stream acquired from a camera as an image tensor of 30 frames per second. The communication unit inputs this image tensor to a convolutional neural network. The neural network outputs a bounding box coordinate of a face of a person in an image and a probability value of an emotion class indicating a facial expression. For example, assume a case where a terminal in a home detects a voice “I am not feeling well today, so I cannot take out the garbage”. The communication unit converts this voice into text data. The communication unit calculates that a probability value of an emotion class of “pain” is 0.85 from a face image acquired at the same time. The communication unit generates a multimodal data packet combining the text data and the emotion probability value. The communication unit transmits this data packet to the AI analysis unit. Assume a case where a voice “The corridor is dark” is input in a common area of an apartment complex. The communication unit includes a current lux value acquired from an illuminance sensor built in the terminal as numerical data in a packet together with voice text. Assume a case where a voice “Tell me the timetable of the nearest bus stop” is input in a public facility. The communication unit adds latitude and longitude coordinate data acquired from a GPS module of the terminal to a packet. The system normalizes these various pieces of input data into a unified data format. The system improves processing efficiency in a subsequent module. The system reduces a data transfer amount to a central server by advanced preprocessing on the edge side. The system reduces a communication delay and realizes a real-time interaction.

The city service linkage unit performs API linkage with a city service and an IoT device, such as a waste management system, public transportation, a meal delivery service, a medical institution, an administrative counter, an air conditioning/ventilation facility, an infrastructure management system, a commercial facility, an educational institution, an event operation system, and a disaster response headquarters, based on a request and a situation of a resident acquired from the communication unit and the sensor linkage unit, and an analysis result of the AI analysis unit, and, for example, arranges an individual collection in cooperation with the waste management system if there is a garbage collection request, automatically notifies a health management center and a family member when a poor physical condition is detected, and arranges a remote medical examination and a nurse dispatch as necessary, automatically guides a real-time bus timetable, a congestion degree, a ride reservation, and an arrangement of a barrier-free vehicle in cooperation with a city transportation system when a resident utters “Tell me the timetable of the nearest bus stop”, automatically executes everything from a reservation of an administrative counter and guidance on necessary documents to support for an online application when a resident utters “I want to perform an administrative procedure”, and collectively supports an event application, a venue reservation, participant recruitment, public relations, guidance on the day, security, cleaning, and cleanup when a resident utters “I want to hold an event in the park”. Specifically, the city service linkage unit functions as an API gateway that manages communication with each external system using an asynchronous messaging queue. The city service linkage unit receives a structured execution command output from the AI analysis unit. The city service linkage unit converts this execution command into a specific data format requested by each external system. For example, assume a case where a command of a garbage collection request is received. The city service linkage unit generates a JSON payload including an ID of a resident, a latitude and longitude coordinate of an address, and a time stamp of a desired collection date and time. The city service linkage unit transmits this payload to a RESTful API of the waste management system via an HTTPS protocol. The city service linkage unit verifies a status code of reception completion returned from the waste management system. The city service linkage unit records a processing result in the data management unit. Assume a case where a poor physical condition is detected. The city service linkage unit evaluates an urgency score output from the AI analysis unit. When the urgency score exceeds a predetermined threshold, the city service linkage unit generates and transmits an electronic medical record linkage message compliant with an HL7 format to a system of a medical institution. At the same time, the city service linkage unit transmits an alert message to a smartphone of a family member via a push notification service. Assume a case where bus timetable guidance is requested. The city service linkage unit issues a query to a GraphQL endpoint of the city transportation system. The city service linkage unit acquires a latest estimated arrival time and congestion degree data of a vehicle using an ID of a designated bus stop as a key. The city service linkage unit inputs the acquired data to a natural language generation model. The city service linkage unit generates a response text “The next bus will arrive in 5 minutes. It is currently empty”. The city service linkage unit returns this response text to the communication unit to synthesize voice. Assume a case where an administrative procedure is requested. The city service linkage unit calls a SOAP interface of a reservation system of an administrative counter. The city service linkage unit acquires an array of free time slots. The city service linkage unit automatically determines an optimal reservation date and time by collating with schedule data of the resident. Assume a case where holding an event is requested. The city service linkage unit starts a transaction in parallel to a facility reservation system, a public relations system, and a security arrangement system. The city service linkage unit commits a process only when all arrangements are completed using a distributed transaction management algorithm. With this configuration, the system automates complex linkage between a plurality of independent systems. The system eliminates a manual adjustment work by a human operator. The system dramatically improves certainty and speed of processing.

The environment and health monitoring unit constantly collects data from various IoT sensors in a city, integrates data from, for example, a fall detection sensor, a heart rate sensor, a blood pressure sensor, a body temperature sensor, a respiration sensor, a sleep sensor, an activity amount sensor, a door opening/closing sensor, a window opening/closing sensor, an illuminance sensor, a temperature sensor, a humidity sensor, an air quality sensor, a carbon dioxide sensor, a noise sensor, a vibration sensor, a water leakage sensor, a smoke sensor, a fire sensor, a gas leak sensor, a congestion degree sensor, a people counting sensor, a face recognition camera, a license plate recognition camera, a traffic volume sensor, a traffic light control sensor, a street light control sensor, a garbage full sensor, a disaster detection sensor, and the like, monitors a health condition and an action of a resident, an operation status of city infrastructure, an environmental change, a disaster sign, a crime risk, and the like in real time, and, for example, immediately calls an API of an emergency transportation service and automatically notifies a family member, a medical institution, a manager, and a neighboring resident when the fall detection sensor detects a fall in a corridor of an apartment complex, automatically notifies a corresponding resident and a facility manager and automatically controls a ventilation system and an air conditioning facility when the air quality sensor falls below a reference value, performs a cause analysis and notifies a resident and a manager as necessary when the noise sensor detects an abnormal value, notifies a user and an operator in real time and automatically proposes and executes a congestion mitigation measure when the congestion degree sensor detects congestion in a public transportation or a commercial facility, and automatically executes an evacuation instruction, an evacuation shelter guidance, provision of a priority service during a disaster, and infrastructure control when the disaster detection sensor detects an earthquake, heavy rain, a flood, a fire, a power outage, and the like. Specifically, the environment and health monitoring unit includes a complex event processing engine that processes streaming data transmitted from millions of IoT sensor nodes deployed in the city in real time. The environment and health monitoring unit buffers time-series numerical data transmitted from each sensor via an MQTT protocol in an in-memory database. The environment and health monitoring unit aggregates data for each certain time window and generates a multidimensional feature vector. For example, assume a case of processing data from a 3-axis acceleration sensor functioning as a fall detection sensor. The environment and health monitoring unit acquires acceleration data of an X-axis, a Y-axis, and a Z-axis at a sampling rate of 100 hertz. The environment and health monitoring unit converts this time-series data into frequency domain data by a fast Fourier transform. The environment and health monitoring unit inputs this data to a support vector machine-based anomaly detection classifier. The classifier discriminates between a normal walking pattern and a fall pattern, and outputs a fall probability score. When the fall probability score exceeds 0.95, the environment and health monitoring unit immediately transmits an interrupt signal to the emergency response unit. Assume a case of processing data from an air quality sensor. The environment and health monitoring unit continuously monitors a concentration value of PM2.5 and a PPM value of carbon dioxide. The environment and health monitoring unit detects that these numerical values exceed a threshold of an environmental reference value. The environment and health monitoring unit automatically transmits a control command for increasing a rotation speed of a ventilation fan to a PLC for controlling an air conditioning facility in a corresponding area. Assume a case of processing data from a noise sensor. The environment and health monitoring unit inputs acquired acoustic data to a sound source separation algorithm. The environment and health monitoring unit identifies a sound source class such as a construction noise, a vehicle noise, or a human scream. When a probability of the scream class is high, the environment and health monitoring unit automatically controls a direction of a nearby security camera to a sound source direction. Assume a case of processing data from a congestion degree sensor. The environment and health monitoring unit inputs depth map data acquired from a stereo camera to a YOLO-based object detection model. The environment and health monitoring unit counts the number of bounding boxes of people in a frame. The environment and health monitoring unit calculates a population density per unit area. When the density exceeds a specified value, the environment and health monitoring unit dynamically switches display content of a digital signage to detour route guidance. Assume a case of processing data from a disaster detection sensor. The environment and health monitoring unit analyzes arrival time difference data of a P wave and an S wave from a seismometer. The environment and health monitoring unit calculates an epicenter and an expected seismic intensity. When the expected seismic intensity exceeds a threshold, the environment and health monitoring unit simultaneously transmits a cutoff signal to gas microcomputer meters in the city. The system executes these advanced data processes in a distributed manner on the edge side and the cloud side. The system establishes an autonomous control loop that is not affected by a network delay. The system technically ensures safety and comfort of a city environment.

The emergency response unit automatically arranges an emergency response, such as emergency transportation, remote medical examination, evacuation guidance, priority service provision during a disaster, infrastructure control, security guard dispatch, volunteer request, family contact, safety confirmation, disaster information provision, disaster recovery support, material distribution, medical support, administrative response, and press release, based on a detection result of the environment and health monitoring unit, utterance content of a resident, and an emergency determination of the AI analysis unit, for example, immediately calls an API of an emergency transportation service and automatically notifies a family member, a medical institution, and an administrative agency when the resident utters “Help” or “Painful” or when a fall detection sensor detects a rapid acceleration change, notifies the resident of a congestion degree and safety information of an evacuation shelter in real time during a disaster, and collectively supports guidance on an evacuation route and a priority service, safety confirmation, family contact, disaster information provision, disaster recovery support, volunteer recruitment, material distribution, medical support, infrastructure recovery, administrative response, press release, and the like. Specifically, the emergency response unit is an event-driven processing module that receives an emergency event trigger transmitted asynchronously from the environment and health monitoring unit and the AI analysis unit. The emergency response unit analyzes an emergency score, location information coordinates, and an event type code included in a payload of the received event. The emergency response unit determines a response procedure by combining a rule-based inference engine and a machine learning-based resource allocation optimization model. For example, assume a case where voice data in which a resident utters “Help” is processed by the AI analysis unit, and an event trigger with an emergency score of 0.99 is generated. The emergency response unit receives this trigger. The emergency response unit immediately transmits a POST request including GPS coordinates of a patient and a predicted symptom code to an API endpoint of an emergency transportation system. The emergency response unit simultaneously searches a registered family contact database. The emergency response unit transmits an automatically generated emergency text message through an SMS gateway. Assume a case where a trigger from a fall detection sensor is received. The emergency response unit acquires past medical history data of a target person from the data management unit. The emergency response unit transfers this medical history data to a tablet terminal of an emergency crew member via an encrypted communication channel. Assume a case where an event trigger of an earthquake occurrence is received during a disaster. The emergency response unit accesses a database of evacuation shelters in the entire city. The emergency response unit acquires a current accommodable number of people of each evacuation shelter and population density data of a disaster-stricken area. The emergency response unit inputs these pieces of data to a linear programming solver. The emergency response unit calculates an optimal evacuation shelter allocation matrix that minimizes a moving distance of residents and does not exceed a capacity of each evacuation shelter. The emergency response unit push-delivers individual evacuation route navigation data to a smartphone of each resident based on this matrix. The emergency response unit simultaneously requests a traffic control system to delegate a control authority of a traffic light. The emergency response unit executes preemption control to fix all traffic lights on a passage route of an emergency vehicle to green. The emergency response unit cooperates with a volunteer management system and automatically transmits a notification of a support request to a registered volunteer living in the vicinity of the disaster-stricken area. The system executes these processes in parallel with multi-threading. The system minimizes a delay in information transmission during an emergency. The system allocates limited resources most efficiently. The system dramatically improves response performance of a computer system for lifesaving and damage mitigation.

The data management unit centrally manages a transition of a health condition, a service usage history, an emergency response history, an occurrence history of an environmental abnormality of each resident, an operation status of infrastructure of the entire city, a disaster occurrence history, a crime occurrence history, a traffic accident history, an event holding history, an administrative procedure history, an energy consumption history, a waste discharge history, a recycling history, a resident satisfaction survey result, and the like, provides appropriate information to a family member, an administrative agency, a medical institution, an infrastructure operator, police, a fire department, an educational institution, a research institution, a private business operator, and the like as necessary, and has functions such as data encryption, access authority management, backup, data erasure, provision of an interface for data analysis, privacy protection, security monitoring, cyber attack countermeasure, legal compliance, anonymization processing, and data distribution management. Specifically, the data management unit adopts a distributed data lake architecture that integrally stores structured data and unstructured data. The data management unit combines a relational database, a time-series database, and a document-oriented database in the right places. The data management unit stores time-series data, such as a transition of a health condition of a resident and an operation status of infrastructure, in a time-series database using a time stamp as a primary key. The data management unit periodically executes a downsampling process on this time-series data to optimize a storage capacity. The data management unit stores transaction data, such as a service usage history and an administrative procedure history, in a relational database that guarantees ACID properties. The data management unit saves unstructured data, such as voice data and image data, in an object storage. The data management unit manages a URI of the saved object as metadata in a database. The data management unit encrypts data using an AES-256 algorithm, which is an advanced encryption standard, when writing data. The data management unit strictly manages an encryption key with a dedicated hardware security module. The data management unit executes token authentication based on an OAuth 2.0 protocol in response to a data access request from an external system. The data management unit evaluates a fine-grained access control list in accordance with a role of a request source. The data management unit technically blocks access to unauthorized data. The data management unit applies a k-anonymization algorithm and differential privacy technology when providing data to a research institution or a private business operator. The data management unit mathematically removes a feature amount capable of identifying an individual from a dataset. The data management unit protects privacy while maintaining usefulness of data. The data management unit continuously collects log data of the entire system. The data management unit inputs this log data to a security information and event management system. The data management unit monitors a sign of a cyber attack and a pattern of unauthorized access in real time using a machine learning-based abnormality detection model. The data management unit automatically updates a firewall rule to block communication when an abnormality is detected. The data management unit executes a data backup process by asynchronous replication between a plurality of geographically separated data centers. The data management unit prevents data loss during a disaster. The system integrates these advanced data management technologies. The system guarantees integrity, confidentiality, and availability of massive city data. The system provides a robust information infrastructure that supports data-driven smart city operation.

The user interface unit provides various interfaces, such as voice, video, touch, gesture, text, AR, VR, web, application, chatbot, and API linkage, so that various users such as a resident, a family member, an administrative official, a medical worker, an infrastructure operator, a security guard, a volunteer, a researcher, a tourist, an event organizer, and a business operator can intuitively use the system, and for example, when a resident inputs a request such as “I want to confirm a health condition of a parent”, “I want to see a waste collection history of my home”, “I want to know an air quality and a noise status in the neighborhood”, “I want to acquire evacuation shelter information during a disaster”, and “I want to apply for participation in an event” from an application, AI extracts necessary information and presents it in an easy-to-understand manner by a graph, a list, a map, a video, voice, an AR display, or the like, and an administrative official can confirm a service usage status and an abnormal tendency of the entire city on a dashboard and utilize them for an additional response, resource allocation, policy making, resident explanation, press release, disaster response headquarters operation, and the like. Specifically, the user interface unit adopts a single-page application architecture in which a front-end rendering engine and a back-end API server are separated. The user interface unit implements a responsive design that dynamically optimizes a layout and an operation component in accordance with a screen size of a user terminal and characteristics of an input device. When a voice input is detected, the user interface unit converts voice into text on a browser using a Web Speech API. The user interface unit transmits this text data to the AI analysis unit. For example, assume a case where a resident inputs a character string “I want to confirm a health condition of a parent” into a text box of a smartphone application. The user interface unit inputs this character string to a natural language processing model. This model performs intent classification and extracts a command “health data inquiry” and a target variable “parent”. The user interface unit issues a GraphQL query to the data management unit based on this extraction result. The user interface unit acquires time-series data of a heart rate and a blood pressure of the parent for the past one week as a JSON array. The user interface unit inputs the acquired data to a data visualization library such as D3.js. The user interface unit generates a DOM element of an interactive line graph and draws it on a screen. For an infrastructure operator wearing AR glasses, the user interface unit superimposes and displays a virtual object indicating an operation parameter of a facility and an abnormal part on a video stream of a real space acquired from a camera. The user interface unit synchronizes a three-dimensional coordinate system of the real space and a coordinate system of the virtual object in real time using a SLAM algorithm. Assume a case where an administrative official accesses a dashboard. The user interface unit accesses a multidimensional data cube in which millions of pieces of sensor data of the entire city are aggregated. The user interface unit executes OLAP processing to aggregate usage statuses by region, by time zone, and by service. The user interface unit renders an aggregation result on a screen as a heat map or a scatter plot. When a user clicks a specific data point on a graph, the user interface unit acquires detailed data by asynchronous communication and performs a drill-down display. Through these processes, the system converts complex and massive city data into a format that can be intuitively understood in accordance with human cognitive ability. The system streamlines a data exploration process of a user. The system technically supports quick and accurate decision-making.

The sensor linkage unit collects data from all IoT sensors in a city in real time, transmits the data to the data management unit and the AI analysis unit, integrates data of, for example, a fall detection sensor, a heart rate sensor, a blood pressure sensor, a body temperature sensor, a respiration sensor, a sleep sensor, an activity amount sensor, a door opening/closing sensor, a window opening/closing sensor, an illuminance sensor, a temperature sensor, a humidity sensor, an air quality sensor, a carbon dioxide sensor, a noise sensor, a vibration sensor, a water leakage sensor, a smoke sensor, a fire sensor, a gas leak sensor, a congestion degree sensor, a people counting sensor, a face recognition camera, a license plate recognition camera, a traffic volume sensor, a traffic light control sensor, a street light control sensor, a waste full sensor, a disaster detection sensor, and the like, and constantly grasps a status of the entire city. Specifically, the sensor linkage unit includes an edge gateway cluster that functions as a data mediation layer between a heterogeneous and diverse IoT sensor group distributed in a city space and a central system. The sensor linkage unit terminates communication protocols of different physical layers and data link layers, such as Bluetooth Low Energy, ZigBee, LoRaWAN, and NB-IoT, used by each sensor. The sensor linkage unit performs protocol conversion of received raw data into a unified internal data structure. The sensor linkage unit analyzes header information of a received data packet. The sensor linkage unit extracts a MAC address and a device ID of a sensor. The sensor linkage unit authenticates validity of data by collating with a device management database. For example, assume a case where vital data is received from a wearable device such as a heart rate sensor and a blood pressure sensor. The sensor linkage unit removes a noise component included in the data using a Kalman filter algorithm. The sensor linkage unit generates a smoothed time-series vector. Assume a case where environmental data from a temperature sensor or a humidity sensor is received. When a missing value is included, the sensor linkage unit complements the data using a linear interpolation algorithm. Assume a case where a high-bandwidth video stream from a face recognition camera or a license plate recognition camera is received. The sensor linkage unit locally executes inference processing on a video frame using a GPU mounted on an edge gateway. The sensor linkage unit transmits only an extracted feature amount vector and text data of a recognition result to a cloud side. The sensor linkage unit thereby dramatically reduces consumption of a network bandwidth. The sensor linkage unit combines data collected from each sensor with a time stamp and geospatial coordinate data. The sensor linkage unit formats them as a spatiotemporal data point. The sensor linkage unit publishes these data points to a distributed messaging system such as Apache Kafka. The sensor linkage unit constructs a streaming pipeline in which the data management unit and the AI analysis unit can consume data asynchronously and in real time as a subscriber. With this configuration, the system realizes high throughput and fault tolerance capable of withstanding high-frequency data transmission from millions of sensors. The system forms a foundation of a digital twin that maps a status of a physical space of a city to a digital space without delay. The system dramatically improves efficiency and reliability of a data collection process.

The AI analysis unit comprehensively analyzes utterance content of a resident, sensor data, a past service usage history, a health condition, environmental information, an operation status of city infrastructure, a disaster risk, a crime risk, a social trend, weather information, traffic information, energy supply and demand, population dynamics, a tourism trend, an economic indicator, an educational indicator, a medical indicator, a welfare indicator, an environmental indicator, disaster statistics, crime statistics, and the like in chronological order using generative AI, and automatically generates and executes a service proposal and information provision optimized for each individual resident, optimal operation of the entire city, a rapid response during a disaster, infrastructure efficiency improvement, social issue resolution, and sustainable city management. For example, it causes generative AI to read a prompt such as “Analyze utterance content of a resident and determine a request, a physical condition change, and urgency”, “Detect a health abnormality, a fall, an air quality deterioration, a congestion degree increase, and a disaster sign from sensor data, and notify a related party as necessary”, “Propose and automatically execute an optimal city service based on an attribute and a past usage history of a resident”, and “Extract a service usage status and an abnormal tendency of the entire city in real time and notify an administrative agency and an infrastructure operator” to perform analysis, determination, and control. Specifically, the AI analysis unit includes an ensemble learning architecture combining a plurality of specialized neural network models and a large language model that supervises them. The AI analysis unit receives multidimensional time-series data and text data streamed from the sensor linkage unit and the data management unit. The AI analysis unit normalizes input data, inputs it to an encoder network corresponding to each modality, and extracts a feature amount vector. The AI analysis unit combines these feature amount vectors and supplies them to an input layer of the large language model. The large language model is composed of a transformer architecture having hundreds of billions of parameters. The large language model calculates a complex dependency relationship between input data using a self-attention mechanism. For example, assume a case where a system prompt “Analyze utterance content of a resident and determine a request, a physical condition change, and urgency” is set. The AI analysis unit inputs voice-recognized text data “My chest hurts” and a time-series vector of a heart rate acquired from a wearable sensor. The large language model outputs structured JSON data of an emergency score of 0.95, a request category “emergency medical care”, and a recommended action “arrangement of an ambulance” from these inputs. Also, assume a case where a prompt “Detect a health abnormality, a fall, an air quality deterioration, a congestion degree increase, and a disaster sign from sensor data, and notify a related party as necessary” is set. The AI analysis unit inputs three-dimensional tensor data of an acceleration sensor and a numerical array of an environmental sensor. The large language model reads an output of an abnormality detection model as a context. The large language model specifies a type of abnormality and an influence range, and generates a list of notification destinations and text of notification content. In the case of a prompt “Propose and automatically execute an optimal city service based on an attribute and a past usage history of a resident”, the AI analysis unit inputs a profile vector and a past action history matrix of a resident. The large language model outputs an ID of a service to be used next and text of a proposal reason in consideration of an output of a collaborative filtering algorithm. In the case of a prompt “Extract a service usage status and an abnormal tendency of the entire city in real time and notify an administrative agency and an infrastructure operator”, the AI analysis unit inputs an aggregated data tensor of the entire city. The large language model specifies a region indicating a statistical outlier and generates a report text including a countermeasure plan. In a learning process of the AI analysis unit, the system uses past city operation data and a determination result of an expert as training data. The system optimizes a weight of a model using fine-tuning and a method of reinforcement learning from human feedback. With this configuration, the system executes advanced inference understanding a context for a complex and ambiguous situation that cannot be handled by a rule-based system. The system dramatically improves an automation level of city operation.

As described above, the system according to the present embodiment centrally and autonomously realizes life support of a resident, health management, public service optimization, disaster response, efficient infrastructure operation, social issue resolution, and sustainable city management on a city scale, and can dramatically enhance QOL improvement of each individual resident, safety, efficiency, and sustainability of the entire city, happiness improvement of the entire society, economic revitalization, environmental conservation, disaster resilience, social inclusion, and innovation creation. Specifically, the system forms an autonomous decentralized feedback loop in which the above-described processing modules closely cooperate. The system converts massive analog information collected from a city space into digital data. The system analyzes the data through tensor calculation in a high-dimensional space. The system directly controls infrastructure equipment and a service provision process in a physical space based on an analysis result. By integrating this cyber-physical system, the system completely replaces a conventional process in which a human operator manually monitors data and makes a determination. The system shortens latency of data processing to a millisecond order. The system dramatically improves a response speed in an emergency. The system utilizes predictive capability of a machine learning model. The system realizes proactive city management that deals with a problem before it occurs. The system executes a dynamic task allocation algorithm of edge computing and cloud computing. The system maximizes utilization efficiency of computing resources while minimizing consumption of a network bandwidth. The system incorporates advanced encryption and anonymization technologies such as federated learning and differential privacy into a data processing pipeline. The system draws out usefulness of big data while mathematically protecting privacy of a resident. The system exhibits a technical effect beyond a framework of mere business efficiency improvement through fundamental improvement of these computer technologies. The system enhances fault tolerance of the entire system. The system ensures scalability. The system provides a robust technical foundation for stably operating a complex system of systems called a smart city.

### Implementation Steps

### Step 1: Start of Data Acquisition of Entire City

Data such as an utterance, an action, a facial expression, a gesture, a touch operation, vital data, an operation status of city infrastructure, an environmental change, a disaster sign, and a crime risk of a resident are acquired in real time from terminals such as a voice recognition robot, a smart speaker, a tablet, a digital signage, a wearable device, a smartphone, an in-vehicle terminal, a public guidance terminal, an electronic bulletin board, and an emergency call button, and various IoT sensors such as a fall detection sensor, a heart rate sensor, a blood pressure sensor, a body temperature sensor, a respiration sensor, a sleep sensor, an activity amount sensor, a door opening/closing sensor, an illuminance sensor, a temperature sensor, a humidity sensor, an air quality sensor, a noise sensor, a vibration sensor, a water leakage sensor, a smoke sensor, a fire sensor, a gas leak sensor, a congestion degree sensor, a people counting sensor, a face recognition camera, a traffic volume sensor, a traffic light control sensor, a street light control sensor, a waste full sensor, and a disaster detection sensor installed in each home, apartment complex, public facility, transportation, medical institution, commercial facility, administrative counter, educational institution, entertainment facility, outdoor plaza, underground mall, parking lot, station, bus stop, taxi stand, sidewalk, intersection, traffic light, street light, waste collection point, evacuation shelter, disaster response base, and the like in a city, and are aggregated in a server in a city center. Specifically, the system initializes a data collection node group deployed in the entire city and establishes a data streaming pipeline. The system executes a handshake protocol for establishing a secure communication channel for each terminal and IoT sensor. The system continuously samples an analog voice signal including an environmental sound from a microphone array of a voice recognition robot or a smart speaker. The system converts the voice signal into digital waveform data in a PCM format using an analog-to-digital converter. The system compresses a video stream acquired from a camera device using an H.265 codec. The system extracts it as an image tensor in units of frames. The system acquires a three-dimensional acceleration vector of an X-axis, a Y-axis, and a Z-axis at a cycle of 100 hertz from an acceleration sensor such as a fall detection sensor and an activity amount sensor. The system periodically polls floating-point numerical data from an environmental sensor such as temperature, humidity, and air quality. The system executes initial preprocessing on these heterogeneous and diverse raw data on an edge device side. The system performs noise removal filtering, missing value interpolation, and data normalization. The system combines the preprocessed data with a time stamp, a device ID, and GPS coordinate data. The system generates a structured data payload in a JSON format. The system transmits this data payload to a cloud server in a city center using an MQTT or gRPC protocol. The system enqueues the received data to a distributed messaging queue through a load balancer on a cloud server side. The system executes sequence control for preventing data loss and order change. By this step, the system captures all events occurring in a physical space of a city in real time as a highly accurate digital data stream that can be processed by a computer. The system continuously constructs a high-quality input dataset for subsequent AI analysis. The system technically improves throughput and reliability of a data collection process.

### Step 2: Multi-layered Analysis and Determination by Generative AI

Generative AI on a server side comprehensively analyzes utterance content of a resident, sensor data, a past service usage history, a health condition, environmental information, an operation status of city infrastructure, a disaster risk, a crime risk, a social trend, weather information, traffic information, energy supply and demand, population dynamics, a tourism trend, an economic indicator, an educational indicator, a medical indicator, a welfare indicator, an environmental indicator, disaster statistics, crime statistics, and the like in chronological order, and determines a request, a health condition, a life pattern, an emergency, a change in a city environment, an operation status of infrastructure, a disaster risk, a crime risk of each individual resident, a service demand and an abnormal tendency of the entire city, and the like in a multifaceted manner. Specific examples of a prompt sentence to be read by generative AI include “Analyze utterance content of a resident and determine a request, a physical condition change, and urgency”, “Detect a health abnormality, a fall, an air quality deterioration, a congestion degree increase, and a disaster sign from sensor data, and notify a related party as necessary”, “Propose and automatically execute an optimal city service based on an attribute and a past usage history of a resident”, and “Extract a service usage status and an abnormal tendency of the entire city in real time and notify an administrative agency and an infrastructure operator”. Specifically, the system inputs the data stream aggregated in Step 1 to an inference pipeline of the generative AI. The system dequeues data from a distributed messaging queue. The system classifies data by modality and inputs it to a corresponding feature amount extraction network. The system converts voice text data into a word embedding vector. The system inputs time-series data of a sensor to a long short-term memory network and extracts a hidden state vector. The system combines these vectors and supplies them to an input layer of a transformer-based large language model. The system simultaneously tokenizes a system prompt and adds it to a head of an input sequence. For example, assume a case where a prompt “Analyze utterance content of a resident and determine a request, a physical condition change, and urgency” is added. The large language model calculates an attention weight between input vectors using a self-attention mechanism. The large language model calculates a probability distribution of each intent class through a softmax function of an output layer. The large language model outputs an intent label with the highest probability and an emergency score in a range of 0.0 to 1.0. Assume a case of a prompt “Detect a health abnormality, a fall, an air quality deterioration, a congestion degree increase, and a disaster sign from sensor data, and notify a related party as necessary”. The large language model evaluates an abnormality score of sensor data as a context. The large language model outputs a JSON object including a classification code indicating a type of abnormality and a list of notification destinations. Assume a case of a prompt “Propose and automatically execute an optimal city service based on an attribute and a past usage history of a resident”. The large language model calculates an inner product of a user profile vector and a service feature amount vector. The large language model outputs an ID of a service with the highest similarity score and text of a proposal reason. Assume a case of a prompt “Extract a service usage status and an abnormal tendency of the entire city in real time and notify an administrative agency and an infrastructure operator”. The large language model analyzes a statistical feature of aggregated data. The large language model outputs coordinate data of a cluster indicating an outlier and text of a countermeasure plan. The system converts these outputs into a standardized command format interpretable by a subsequent processing module. By this step, the system automatically derives an executable action from complex multidimensional data. The system executes information processing at a speed and accuracy that surpass a human cognitive process while imitating it.

### Step 3: City Service Linkage and Automatic Execution

The city service linkage unit performs API linkage with a city service and an IoT device, such as a waste management system, public transportation, a meal delivery service, a medical institution, an administrative counter, an air conditioning/ventilation facility, an infrastructure management system, a commercial facility, an educational institution, an event operation system, and a disaster response headquarters, based on an analysis result of generative AI, and automatically selects and executes an optimal city service in accordance with a request and a health condition of a resident, an environmental change, and a disaster risk. For example, it arranges individual collection when there is a waste collection request, automatically notifies a health management center and a family member when a poor physical condition is detected, arranges remote medical examination and nurse dispatch as necessary, and collectively realizes traffic guidance, a boarding reservation, arrangement of a barrier-free vehicle, a reservation and an online application for an administrative procedure, event holding support, evacuation guidance and priority service provision during a disaster, and the like. Specifically, the system receives the standardized command generated in Step 2 and starts a specific control transaction for an external system. The system analyzes an action code included in the command. The system acquires a URL of an API endpoint and an authentication token of a corresponding external system from a database. For example, assume a case where a waste collection arrangement command is received. The system constructs an HTTP POST request including address coordinates of a resident and a desired collection date and time. The system transmits this request to a REST API of a waste management system. The system confirms a response code 200 and completes the process. Assume a case where a poor physical condition detection command is received. The system evaluates an emergency score. When the score exceeds a threshold, the system push-transmits a real-time alert message to a system of a health management center via WebSocket. The system simultaneously calls an API of a remote medical examination system and generates a session URL of a video call. The system transmits this URL to a terminal of the resident and displays it on a screen. Assume a case where a traffic guidance command is received. The system issues a query to a GraphQL endpoint of a city transportation system. The system acquires a shortest route node list from a current location to a destination and GPS coordinates of a current location of each bus. The system integrates these pieces of data and generates JSON data for navigation. The system transmits this data to a smartphone of the resident. Assume a case where an administrative procedure command is received. The system calls a SOAP interface of an administrative counter system. The system transmits an XML payload including authentication information of the resident and application content, and acquires a reception number. Assume a case where an event holding support command is received. The system transmits an API request in parallel to each system of facility reservation, public relations, and security. The system executes distributed transaction processing that confirms an overall arrangement only when an approval response is obtained from all systems. Assume a case where an evacuation guidance command during a disaster is received. The system acquires accommodation status data of each evacuation shelter. The system calculates an optimal evacuation shelter based on location information of the resident. The system transmits coordinate data of an evacuation route to a terminal of the resident. By this step, the system programmatically automates complex linkage between a plurality of independent systems. The system eliminates a delay and an error due to a manual operation, and technically optimizes a provision process of a city service.

### Step 4: Environment and Health Monitoring and Abnormality Detection

The environment and health monitoring unit constantly collects data from various IoT sensors in the city, detects a health abnormality, a fall, an air quality deterioration, a noise abnormality, a congestion degree increase, a disaster sign, a crime risk, and the like in real time through analysis using generative AI, automatically notifies the resident himself/herself, a family member, an administrative agency, a medical institution, an infrastructure operator, a security guard, a volunteer, and the like as necessary, and automatically arranges an emergency response, such as emergency transportation, remote medical examination, air conditioning/ventilation control, evacuation guidance, infrastructure control, security guard dispatch, volunteer request, family contact, safety confirmation, disaster information provision, disaster recovery support, relief supply distribution, medical support, administrative response, and press release. Specific examples of a prompt sentence to be read by the generative AI include “Detect a health abnormality, a fall, an air quality deterioration, a congestion degree increase, and a disaster sign from sensor data, and notify a related party as necessary” and “Automatically arrange an emergency response when an abnormality is detected”. Specifically, the present system continuously executes a real-time abnormality detection algorithm for streaming data in the environment and health monitoring unit. The present system extracts time-series data received from each IoT sensor by a sliding window method for each certain time window. The present system calculates a statistical feature amount from the extracted data window and generates a feature amount vector. The present system inputs this feature amount vector to a pre-trained autoencoder network. The present system calculates a reconstruction error between the input vector and a reconstruction vector. The present system sets a flag as an abnormal event when the reconstruction error exceeds a dynamically set threshold value. The present system inputs data of this abnormal event to the present generative AI. The present system causes the data to be processed together with a prompt “Detect a health abnormality, a fall, an air quality deterioration, a congestion degree increase, and a disaster sign from sensor data, and notify a related party as necessary”. The present generative AI classifies the type and urgency of the abnormality and outputs a list of specific response actions. For example, when a fall is detected, the present generative AI outputs “arrangement of emergency transportation” and “notification to a family member” as actions. The present system triggers the present emergency response unit in accordance with a prompt “Automatically arrange an emergency response when an abnormality is detected”. The present emergency response unit calls an API of an emergency transportation system and transmits GPS coordinates of a target person and predicted symptom data. At the same time, the present emergency response unit transmits an alert message to a smartphone of a family member through an SMS gateway. When an air quality deterioration is detected, the present system transmits a control command for maximizing a ventilation mode to a PLC of an air conditioning facility in a corresponding area using an MQTT protocol. When a congestion degree increase is detected, the present system transmits image data of a detour route to a display controller of digital signage to switch a display. When a disaster sign is detected, the present system transmits an activation command to a siren control system of the entire city. At the same time, the present system simultaneously transmits a text message of an evacuation instruction to terminals of all residents via a push notification service. By this step, the present system detects a change or an abnormality in a city environment with a delay in milliseconds. The present system immediately executes a physical response action without human intervention. The present system establishes an autonomous control loop for preventing the spread of damage.

### Step 5: Data Management, Information Provision, and Continuous Optimization

The data management unit centrally manages a transition of a health condition for each resident, a service usage history, an emergency response history, an occurrence history of an environmental abnormality, an operation status of city infrastructure of the entire city, a disaster occurrence history, a crime occurrence history, a traffic accident history, an event holding history, an administrative procedure history, an energy consumption history, a waste discharge history, a recycling history, a resident satisfaction survey result, and the like, and provides necessary information in an easy-to-understand manner to various users such as a resident, a family member, an administrative official, a medical worker, an infrastructure operator, a security guard, a volunteer, a researcher, a tourist, an event organizer, and a business operator through the user interface unit using various interfaces such as voice, video, touch, gesture, text, AR, VR, web, application, chatbot, and API linkage, and the generative AI comprehensively analyzes an attribute of a resident, a past usage history, a health condition, a life pattern, an environmental change, a status of the entire city, an infrastructure operation status, a disaster risk, a crime risk, a service demand, resident satisfaction, a policy effect, a social trend, weather information, traffic information, energy supply and demand, population dynamics, a tourism trend, an economic indicator, an educational indicator, a medical indicator, a welfare indicator, an environmental indicator, disaster statistics, crime statistics, and the like, and continuously optimizes a service proposal and information provision optimized for each individual resident, optimal operation of the entire city, a rapid response during a disaster, infrastructure efficiency improvement, social issue resolution, and sustainable city management. Specific examples of a prompt sentence to be read by the generative AI include “Optimize a service proposal and a city operation policy from the next time onward based on an attribute and a usage history of a resident and a status of the entire city”. Specifically, the present system persists all transaction data, sensor logs, and AI inference results from Step 1 to Step 4 in a distributed database of the present data management unit. The present system inputs these accumulated pieces of history data to a batch processing pipeline. The present system executes an ETL process to cleanse the data and loads the data into a data warehouse. The present system issues an SQL query to this data warehouse in response to a request from the user interface unit. The present system converts the extracted data into a JSON format and passes the data to a front-end drawing library to generate a graph and a dashboard. For continuous optimization, the present system uses the accumulated data as a dataset for retraining and fine-tuning of the present generative AI. The present system sets a prompt “Optimize a service proposal and a city operation policy from the next time onward based on an attribute and a usage history of a resident and a status of the entire city”. The present system uses an acceptance history of a resident for a past service proposal as a reward signal. The present system executes a reinforcement learning algorithm. The present system updates parameters of the present generative AI to improve the accuracy of the proposal. The present system inputs time-series data of an energy consumption history and a traffic congestion history of the entire city to an autoregressive integrated moving average model or a Prophet model. The present system generates a future demand forecast curve. Based on this forecast curve, the present system dynamically recalculates an operation schedule of infrastructure facilities and a diagram of public transportation. The present system feeds back the recalculated parameters to each infrastructure management system. The present system inputs text data of a resident satisfaction survey to a natural language processing model. The present system executes topic modeling to extract a cluster of potential dissatisfactions and requests. The present system automatically generates information of the extracted cluster as a policy planning support report for an administrative official. By this step, the present system realizes a self-evolving mechanism in which the system itself learns from experience and improves performance over time. The present system provides a computer system with adaptability to environmental changes, which is impossible with a static rule-based system. The present system achieves technical improvement through integration of data management and machine learning.

### Specific Use Case

For example, when an elderly person living in an apartment complex in a city utters “I am not feeling well today, so I cannot take out the garbage” to a voice recognition robot installed in a living room, the communication unit acquires this utterance in real time and transmits it to an AI analysis unit. The AI analysis unit causes the generative AI to read a prompt “Analyze utterance content of a resident and determine a request, a physical condition change, and urgency”, and simultaneously extracts a poor physical condition and a garbage collection request. The city service linkage unit performs API linkage with a waste management system, automatically arranges individual garbage collection to the resident's home, notifies a health management center or a family member of the poor physical condition as necessary, and further arranges remote medical examination or nurse dispatch when deterioration of a health condition is suspected. For example, when the same resident utters “I want to go shopping”, the AI analysis unit causes the generative AI to read a prompt “Analyze utterance content of a resident and extract a request for shopping support”, cooperates with a nearby commercial facility or an online shopping site, and collectively arranges from a product proposal to ordering and delivery. For example, when an utterance “Tell me the timetable of the nearest bus stop” is made to a guidance terminal of a public facility, the AI analysis unit causes the generative AI to read a prompt “Extract a usage request for public transportation from utterance content of a resident and provide an optimal timetable and transfer guidance”, and automatically guides real-time bus times, a congestion status, a boarding reservation, and arrangement of a barrier-free vehicle in cooperation with a city transportation system. For example, when an IoT sensor detects a fall in a corridor of an apartment complex, a sensor linkage unit causes the generative AI to read a prompt “Detect a fall or a health abnormality from sensor data and notify a family member or an administrative agency as necessary”, and when it is determined that urgency is high, the emergency response unit calls an API of an emergency transportation service and automatically notifies a family member, a medical institution, a manager, and a neighboring resident. For example, when an air quality sensor falls below a reference value, the AI analysis unit causes the generative AI to read a prompt “Propose ventilation or use of an air purifier to a resident for whom an air quality deterioration is detected”, automatically notifies a corresponding resident or a facility manager, and automatically controls a ventilation system or an air conditioning facility as necessary. For example, when a congestion degree sensor detects congestion in a commercial facility, the AI analysis unit causes the generative AI to read a prompt “When a congestion degree increase is detected, notify a user or an operator and propose a congestion mitigation measure” and notifies a user or an operator in real time, and automatically proposes and executes a congestion mitigation measure such as admission restriction, staggered use, extra flight operation, guidance, and coupon distribution. For example, when a disaster detection sensor detects an earthquake, heavy rain, a flood, a fire, a power outage, or the like, the AI analysis unit causes the generative AI to read a prompt “When a disaster sign is detected, automatically execute an evacuation instruction, evacuation shelter guidance, and provision of a priority service during a disaster”, notifies a resident of a congestion status and safety information of an evacuation shelter in real time, and collectively supports guidance of an evacuation route and a priority service, safety confirmation, family contact, disaster information provision, disaster recovery support, volunteer recruitment, relief supply distribution, medical support, infrastructure recovery, administrative response, press release, and the like. For example, when a resident or a family member inputs a request such as “I want to confirm a health condition of my parent”, “I want to see a garbage collection history of my home”,

“I want to know an air quality and a noise status in the neighborhood”, “I want to acquire evacuation shelter information during a disaster”, or “I want to apply for participation in an event” from an application, the AI analysis unit causes the generative AI to read a prompt “Extract necessary information in response to an inquiry from a resident or a family member and present it in an easy-to-understand manner”, extracts necessary information, and presents it in an easy-to-understand manner using a graph, a list, a map, a video, voice, an AR display, or the like. Specific examples of a prompt sentence to be read by the generative AI necessary for carrying out the present invention include “Analyze utterance content of a resident and determine a request, a physical condition change, and urgency”, “Detect a health abnormality, a fall, an air quality deterioration, a congestion degree increase, and a disaster sign from sensor data, and notify a related party as necessary”, “Propose and automatically execute an optimal city service based on an attribute and a past usage history of a resident”, “Extract a service usage status and an abnormal tendency of the entire city in real time and notify an administrative agency or an infrastructure operator”, and “Extract necessary information in response to an inquiry from a resident or a family member and present it in an easy-to-understand manner”. Specifically, the present system processes these various use cases in parallel on a single integrated architecture. The present system converts an analog voice acquired from a microphone of a voice recognition robot into a digital waveform. The present system converts this waveform data into a mel-spectrogram feature amount, inputs it to a voice recognition model, and extracts a text character string. The present system inputs this text character string to the present generative AI. The present generative AI tokenizes the input text and maps it to a multidimensional vector space. The present generative AI analyzes a context using a self-attention mechanism and outputs an intent classification score and an entity extraction result. For example, in response to an input “I am not feeling well today, so I cannot take out the garbage”, the present generative AI outputs a flag of “poor physical condition” and a command code of “garbage collection request”. Based on this output, the present system transmits an HTTP POST request to an API of a waste management system and writes a collection schedule in a database. At the same time, the present system calls an API of a health management system and sets an alert flag. In response to an input “I want to go shopping”, the present generative AI extracts an intent of “shopping support” and issues an SQL query to a past purchase history database. The present system generates a JSON array of a recommended product list based on the acquired history data, transmits it to an API of an EC site, and adds a product to a cart. In response to an input “Tell me the timetable of the nearest bus stop”, the present system acquires GPS coordinates of a terminal. The present system transmits coordinate data to an API of a city transportation system and acquires timetable data of a bus stop within a radius of 500 meters. The present system inputs this data to a natural language generation model and generates text for voice synthesis. When receiving acceleration data from a fall detection sensor, the present system performs a fast Fourier transform on the data to extract a frequency component. The present system inputs it to an abnormality detection model to calculate a fall probability score. When the score exceeds a threshold value, the present system immediately calls an API of an emergency transportation system. When PM2.5 concentration data from an air quality sensor exceeds a threshold value, the present system transmits a ventilation command to a PLC of an air conditioning facility using an MQTT protocol. When a camera image from a congestion degree sensor is analyzed by a YOLO model and a population density exceeds a threshold value, the present system draws an image of a detour route on digital signage. When receiving seismic wave data from a disaster detection sensor, the present system accesses a database of an evacuation shelter and calculates an available capacity and a distance from a current location. The present system generates a coordinate list of an optimal evacuation route and pushes a notification to a smartphone of a resident. In response to an information inquiry request from an application, the present system acquires corresponding time-series data from a database. The present system inputs the data to a visualization library, generates a DOM element of a graph, and renders it on a screen. In these processes, the present system dynamically inserts each prompt sentence as a system prompt into a context window of the present generative AI. The present system controls an inference direction of the model. With this configuration, the present system executes complex situation judgment and device control in real time without human intervention. The present system realizes complete automation and optimization of city infrastructure using computer technology.

The specific processing unit 290 sends the results of specific processing obtained in Example 1 or Application Example 1 described above to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence).

An example of the data generation model 58 is a generative AI such as ChatGPT (registered trademark) (Internet search <URL: https://openai.com/blog/chatgpt>).

The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart device 14.

### Second Embodiment

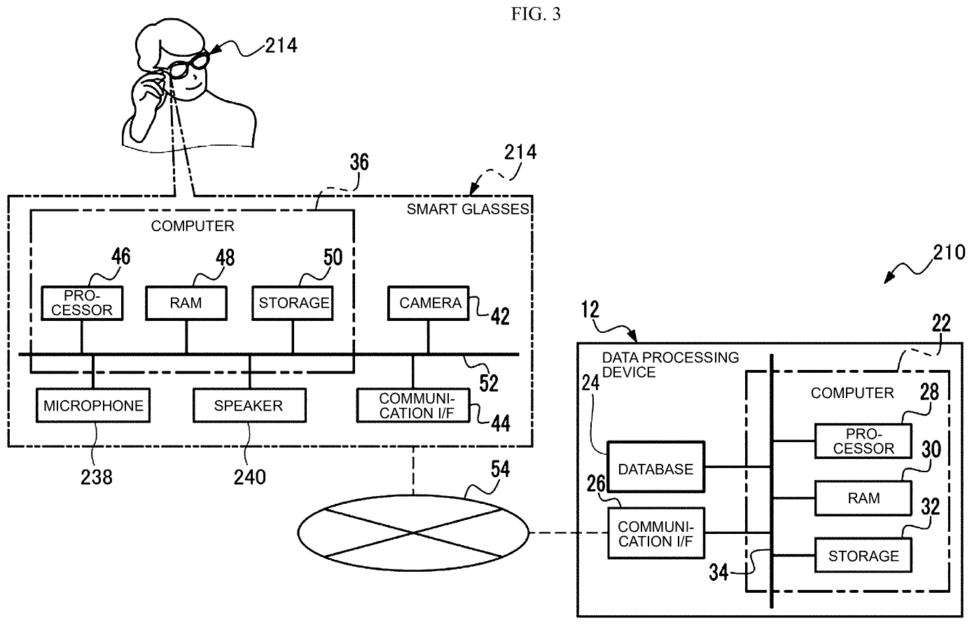

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, camera 42, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

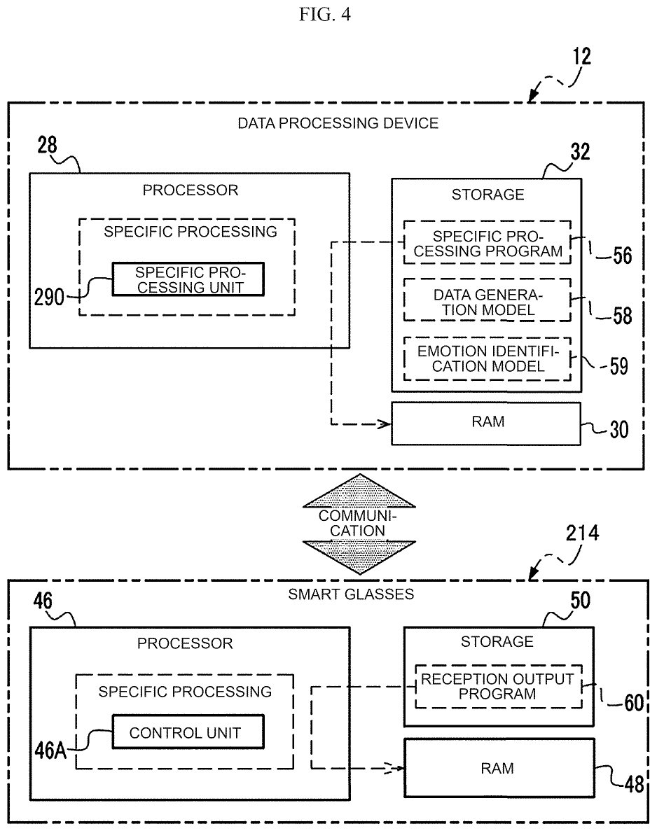

FIG. 4 shows an example of the main functions of the data processing device 12 and smart glasses 214. As shown in FIG. 4, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart glasses 214, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48. The smart glasses 214 may also have similar data generation models and emotion identification models as the data generation model 58 and emotion identification model 59, and perform the same processing as the specific processing unit 290 using these models.

Next, specific processing by the specific processing unit 290 of the data processing device 12 will be described. Each part of the system described below is realized by the data processing device 12 and the smart glasses 214. In the following description, the data processing device 12 is referred to as a “server,” and the smart glasses 214 are referred to as a “terminal.”

### Example 1

Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart glasses 214.

### Third Embodiment

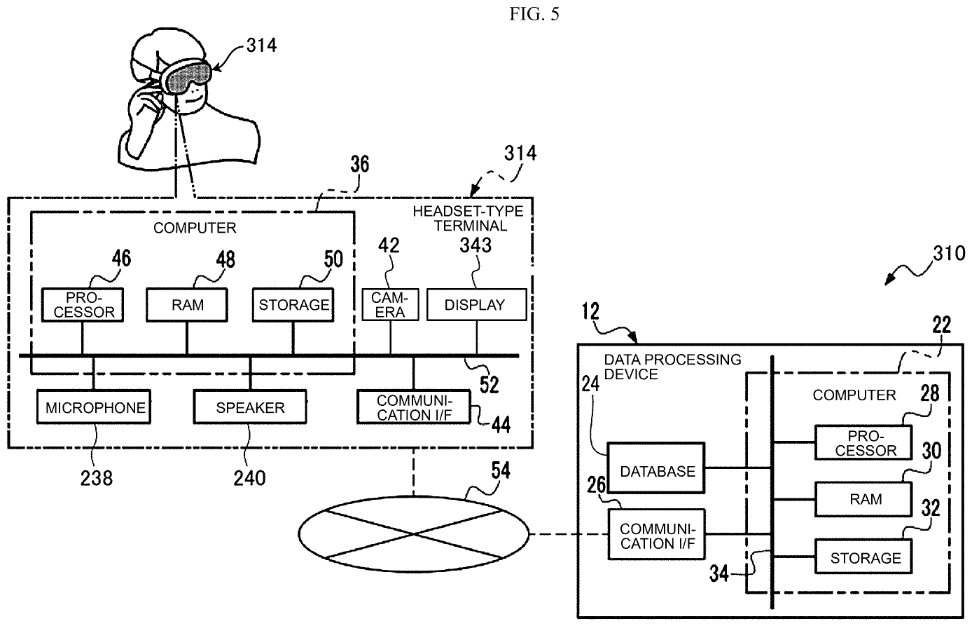

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, display 343, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

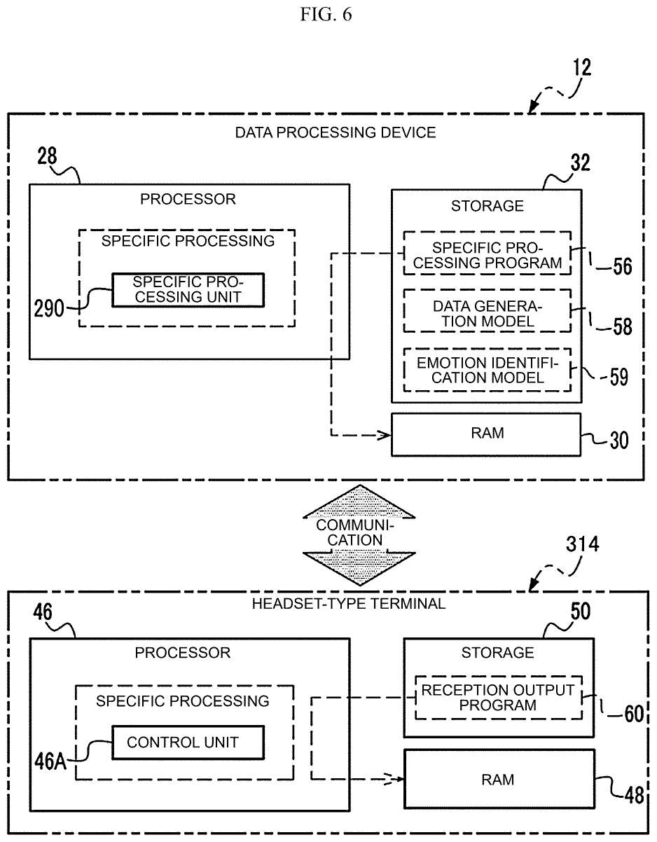

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the headset-type terminal 314, reception output processing is performed by the processor 46.

The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the headset-type terminal 314. In the following description, the data processing device 12 is referred to as a “server,” and the headset-type terminal 314 is referred to as a “terminal.”

(Example 1) Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

In the above embodiments, an example form where specific processing is performed by the data processing device 12 was described, but the technology disclosed herein is not limited to this, and specific processing may be performed by the headset-type terminal 314.

### Fourth Embodiment

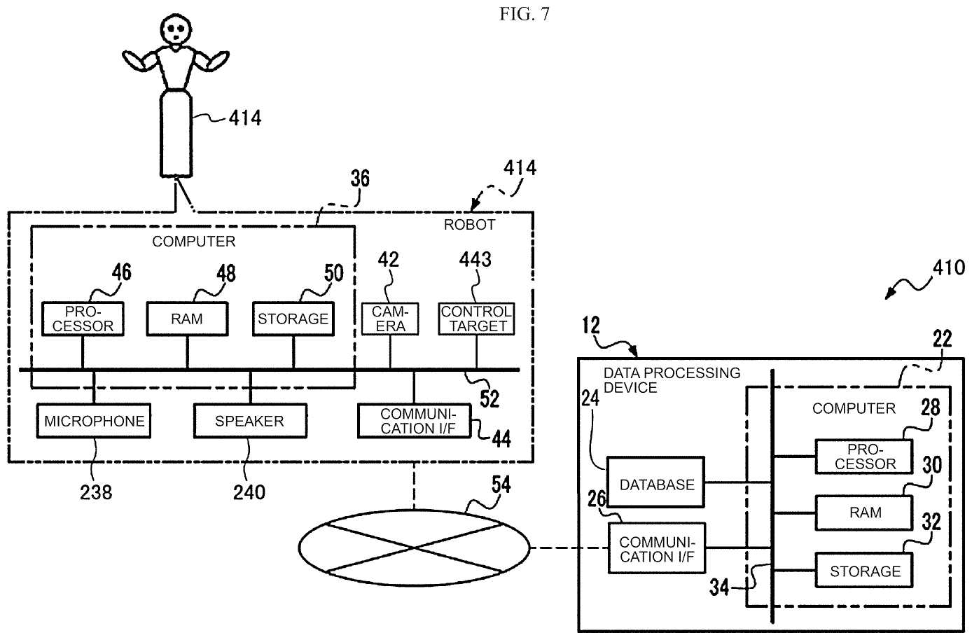

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, control target 443, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

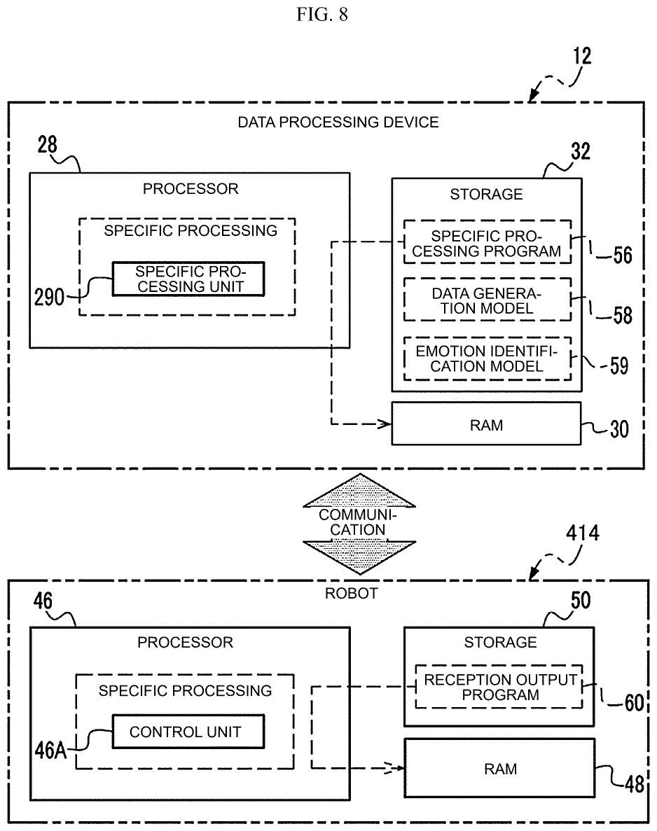

FIG. 8 shows an example of the main functions of the data processing device 12 and the robot 414. As shown in FIG. 8, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the robot 414, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the robot 414. In the following description, the data processing device 12 is referred to as a “server,” and the robot 414 is referred to as a “terminal.”

(Example 1) Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

In the above embodiments, an example form where specific processing is performed by the data processing device 12 was described, but the technology disclosed herein is not limited to this, and specific processing may be performed by the robot 414.

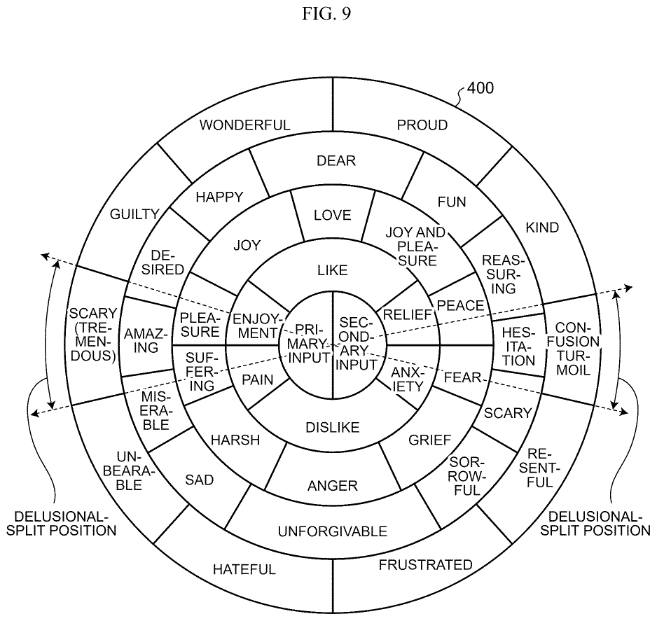

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

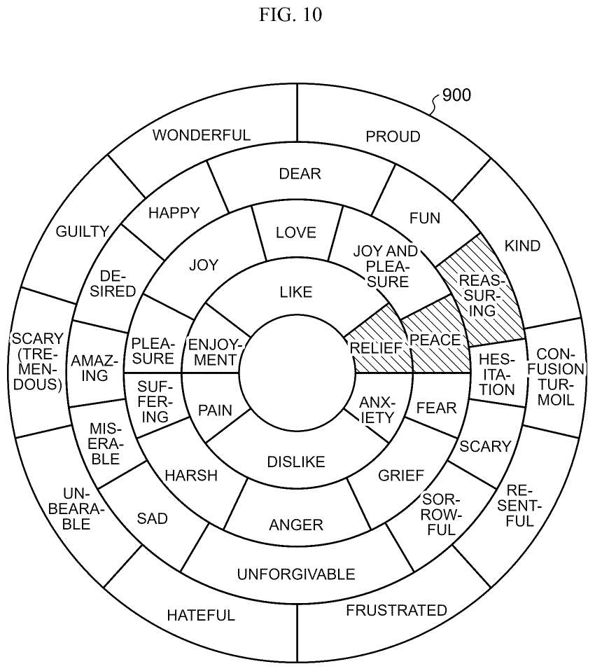

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above, the system according to the present disclosure has been described mainly on the functions of the data processing device 12, but the system according to the present disclosure is not necessarily mounted on a server. The system according to the present disclosure may be implemented as a general information processing system. The present disclosure may be implemented, for example, as a software program running on a personal computer, or an application running on a smartphone or the like. The method according to the present disclosure may be provided to the user in the form of SaaS (Software as a Service).

In the above embodiments, an example form where specific processing is performed by a single computer 22 was described, but the technology disclosed herein is not limited to this, and distributed processing for specific processing by multiple computers including the computer 22 may be performed. For example, the data generation model 58 may be provided in an external device of the data processing device 12, and the external device may generate data corresponding to the input data.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned.

Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

Note that the following supplementary notes are further disclosed regarding the above description.

### Supplementary Note 1

A comprehensive city life support system comprising: a communication unit; a city service linkage unit; an environment and health monitoring unit; an emergency response unit; a data management unit; and a user interface unit, wherein the communication unit is configured to acquire an utterance, an action, environmental information, vital data, and the like of a resident in real time using a communication terminal and an IoT sensor installed in each home, apartment complex, public facility, transportation, medical institution, commercial facility, administrative counter, and the like in a city, and analyze a request, a health condition, a life pattern, an emergency, a change in a city environment, and the like of each individual resident in a multifaceted manner using generative AI; the city service linkage unit is configured to perform API linkage with a city service and an IoT device, such as a waste management system, public transportation, a meal delivery service, a medical institution, an administrative counter, an air conditioning/ventilation facility, and an infrastructure management system, based on an analysis result acquired from the communication unit, and automatically select and execute an optimal city service in accordance with the request and the health condition of the resident and an environmental change; the environment and health monitoring unit is configured to constantly collect data from various sensors in the city, detect a health abnormality, a fall, an air quality deterioration, a congestion degree increase, a disaster sign, and the like in real time using generative AI, and automatically notify a family member, an administrative agency, a medical institution, an infrastructure operator, and the like as necessary; the emergency response unit is configured to automatically arrange an emergency response, such as emergency transportation, remote medical examination, evacuation guidance, and priority service provision during a disaster, based on a detection result of the environment and health monitoring unit and utterance content of the resident; the data management unit is configured to safely and efficiently record and manage a resident status of the entire city, a service usage history, health data, environmental data, an emergency response history, and the like in chronological order; and the user interface unit is configured to provide various interfaces, such as voice, video, touch, and text, so that the resident, a family member, and an administrative official can intuitively use the system, and automatically generate individually optimized information presentation and a service proposal based on an attribute and a past usage history of the resident.

### Supplementary Note 2

The comprehensive city life support system according to Supplementary Note 1, wherein the city service linkage unit is configured to comprehensively analyze utterance content of a resident, sensor data, a past service usage history, a health condition, environmental information, an operation status of city infrastructure, and the like using generative AI, individually optimize and automatically propose and execute various city services, such as waste collection, public transportation guidance/reservation, meal delivery/shopping support, medical/health support, air conditioning/ventilation control, evacuation guidance during a disaster, event guidance, and administrative procedure support, for each individual resident, further link and schedule a plurality of services simultaneously, and dynamically control service content and a priority flexibly in accordance with a life pattern of the resident and a status change of the entire city.

### Supplementary Note 3

The comprehensive city life support system according to Supplementary Note 1, wherein the environment and health monitoring unit is configured to analyze vital data, fall detection data, air quality data, noise data, congestion degree data, illuminance data, disaster-related data, and the like acquired from various sensors in the city in chronological order using generative AI, automatically notify the resident himself/herself, a family member, an administrative agency, a medical institution, an infrastructure operator, and the like when an abnormality such as a health abnormality, a fall, an air quality deterioration, a noise abnormality, a congestion degree increase, and a disaster sign is detected in real time, automatically arrange an emergency response, such as emergency transportation, remote medical examination, air conditioning/ventilation control, evacuation guidance, and infrastructure control, as necessary, and further extract an abnormal tendency and a risk of the entire city to notify an administrative agency and an infrastructure operator in real time.

What is claimed is:

1. A system comprising:
circuitry configured to:
receive, via a communication interface coupled to a packet-switched network, a multidimensional data stream from a plurality of edge nodes and sensor devices distributed across a physical environment, and ingest and aggregate the multidimensional data stream in real time using a distributed stream processing engine;
input the aggregated data stream to a digital twin model of the physical environment, the digital twin model comprising a graph neural network modeling physical connection relationships among infrastructure components and state dynamics of entities in the environment, and generate an optimal resource allocation matrix and infrastructure control commands as output;
apply a natural language processing model to dialogue data received from a terminal device to recognize a request identifier and a state change indicator from the dialogue data, and generate a service selection data record by applying a generative neural network model to the request identifier and a preference profile record associated with a user identifier;
monitor sensor data streams for anomaly signals by applying an autoencoder model pre-trained on normal operating conditions, and generate an emergency response command when a reconstruction error exceeds an emergency threshold parameter; and
transmit the infrastructure control commands, the service selection data record, and activity history data to terminal devices via the communication interface.

2. The system according to claim 1, wherein the circuitry is configured to
define edge nodes of a first processing layer, fog gateway nodes of a second processing layer, and cloud server nodes of a third processing layer in a hierarchically integrated distributed processing architecture, apply lightweight inference at edge nodes to generate compact feature vectors, aggregate compact feature vectors at fog gateway nodes, and execute full model inference on the aggregated data at cloud server nodes.

3. The system according to claim 2, wherein the circuitry is configured to
receive a multidimensional data stream from millions of sensor devices via the communication interface, distribute incoming data stream records to stream processing nodes via a load balancer, and apply a distributed stream processing engine to ingest and aggregate data stream records in real time prior to input to the digital twin model.

4. The system according to claim 1, wherein the circuitry is configured to
apply the graph neural network of the digital twin model to a graph structure comprising infrastructure component nodes and directed dependency edges, execute message passing operations between adjacent nodes to propagate state update vectors, and generate the resource allocation matrix by applying an optimization objective function to the propagated node state vectors.

5. The system according to claim 4, wherein the circuitry is configured to
update node state vectors in the digital twin model in real time as new sensor data is received from corresponding infrastructure component sensor devices, and generate updated infrastructure control commands by re-applying the optimization objective function to the updated resource allocation matrix at configurable intervals.

6. The system according to claim 1, wherein the circuitry is configured to
apply a transformer-based natural language processing model with positional encoding to dialogue data token sequences to extract named entity labels comprising request type identifiers and preference indicators, and compute a dialogue embedding vector for each dialogue session.

7. The system according to claim 6, wherein the circuitry is configured to
generate the service selection data record by applying a retrieval-augmented generation process comprising retrieving service catalog records from a vector database by cosine similarity search using the dialogue embedding vector, and generating a service selection sequence by autoregressive decoding conditioned on the retrieved service catalog records and the preference profile record.

8. The system according to claim 7, wherein the circuitry is configured to
transmit service execution commands to external service endpoint systems via the communication interface based on the service selection data record, receive execution confirmation records from the external service endpoint systems, and store the execution confirmation records in an activity history data structure in the storage device associated with the user identifier.

9. The system according to claim 1, wherein the circuitry is configured to
apply the autoencoder model to time-series sensor data arrays by computing reconstruction error values between input sensor data arrays and autoencoder output arrays, detect an anomaly signal when a reconstruction error value exceeds an anomaly threshold parameter, and classify the anomaly signal using a multi-class classification model to generate an anomaly type identifier.

10. The system according to claim 9, wherein the circuitry is configured to
generate the emergency response command by applying the generative neural network model to the anomaly type identifier, the current resource allocation matrix, and emergency response procedure records retrieved from a vector database, and transmit the emergency response command to designated terminal devices and emergency service endpoint systems via the communication interface.

11. The system according to claim 1, wherein the circuitry is configured to
store activity history data comprising service execution records, dialogue session records, and sensor event records in a longitudinal data structure in the storage device associated with each user identifier, and apply the generative neural network model to the longitudinal data structure to update the preference profile record by extracting preference patterns from historical activity records.

12. The system according to claim 11, wherein the circuitry is configured to
apply a pattern deviation detection algorithm to the longitudinal activity data structure by comparing recent activity frequency metrics against historical baseline activity frequency distributions, and generate an inactivity alert signal when activity frequency metrics fall below an inactivity threshold parameter for a configurable time window.

13. The system according to claim 1, wherein the circuitry is configured to
transmit activity history data to terminal devices associated with designated contact user identifiers via the communication interface for remote monitoring of current state information, and enforce a privacy access control policy restricting visibility of activity history data fields to authorized user identifiers.

14. The system according to claim 1, wherein the circuitry is configured to
update the digital twin model by generating training pairs from historical infrastructure control command records and corresponding validated infrastructure state outcome records, and fine-tune the graph neural network on the training pairs to reduce prediction error on infrastructure state transitions.

15. The system according to claim 1, wherein the circuitry is configured to
synchronize the digital twin model state with the physical environment by applying sensor data fusion across heterogeneous sensor modalities comprising numerical time-series arrays, binary state arrays, and image tensor data, and maintaining a consistent virtual state representation for each infrastructure component node.

16. The system according to claim 1, wherein the circuitry is configured to
encrypt data transmissions between edge nodes, fog gateway nodes, and cloud server nodes using a transport layer security protocol, and authenticate each node using a digital certificate prior to accepting data stream records for ingestion by the distributed stream processing engine.

17. The system according to claim 1, wherein the edge nodes and sensor devices are distributed across a plurality of physical infrastructure locations, the graph neural network models infrastructure component dependency relationships, and the resource allocation matrix represents optimal allocation of infrastructure processing capacity across the physical environment.

18. A system comprising:
circuitry configured to:
receive, via a communication interface coupled to a packet-switched network, a multidimensional data stream from edge nodes and sensor devices in a hierarchically distributed processing architecture comprising edge, fog, and cloud layers;
input aggregated data to a digital twin model comprising a graph neural network modeling physical connection relationships, and generate a resource allocation matrix and infrastructure control commands;
apply a transformer-based natural language processing model to dialogue data to extract request identifiers, and generate a service selection data record by retrieval-augmented generation from a vector database conditioned on a preference profile record;
apply an autoencoder anomaly detection model to sensor data arrays, and generate an emergency response command when a reconstruction error exceeds an emergency threshold; and
transmit infrastructure control commands, service selection data records, and activity history data to terminal devices via the communication interface.

19. The system according to claim 18, wherein the circuitry is configured to
apply a pattern deviation detection algorithm to a longitudinal activity data structure to generate inactivity alert signals when activity frequency metrics fall below a threshold, enforce a privacy access control policy for remote monitoring access to activity history data, and update the graph neural network of the digital twin model by fine-tuning on historical infrastructure state training pairs.

20. A method comprising:
receiving, via a communication interface coupled to a packet-switched network, a multidimensional data stream from a plurality of edge nodes and sensor devices distributed across a physical environment, and ingesting and aggregating the multidimensional data stream using a distributed stream processing engine;
inputting the aggregated data stream to a digital twin model comprising a graph neural network modeling physical connection relationships among infrastructure components, and generating an optimal resource allocation matrix and infrastructure control commands;
applying a natural language processing model to dialogue data received from a terminal device to recognize a request identifier, and generating a service selection data record by applying a generative neural network model conditioned on a preference profile record and context data retrieved from a vector database;
monitoring sensor data streams for anomaly signals using an autoencoder model, and generating an emergency response command when a reconstruction error exceeds an emergency threshold parameter; and
transmitting the infrastructure control commands, the service selection data record, and activity history data to terminal devices via the communication interface.
